(19) United States
(12) Patent Application Publication
(10) Pub. No.: US 2023/0413819 A1
(43) Pub. Date: Dec. 28, 2023

(54) METHODS FOR CONTROLLING FLEA AND TICK INFESTATIONS IN A MAMMAL

(71) Applicant: In the Bowl Animal Health, Inc., Nashville, TN (US)

(72) Inventors: Robin S. Readnour, Nashville, TN (US); Kevin E. Willard, Knightstown, IN (US); Joseph R. Winkle, Carmel, IN (US)

(21) Appl. No.: 18/464,233

(22) Filed: Sep. 10, 2023

Related U.S. Application Data
(63) Continuation of application No. PCT/US2022/019913, filed on Mar. 11, 2022.
(60) Provisional application No. 63/159,801, filed on Mar. 11, 2021.

(51) Int. Cl. A01N 43/22; A01N 43/76; A01P 17/00

(52) U.S. Cl. CPC A01N 43/22; A01N 43/76; A01P 17/00

(57) ABSTRACT

Orally administrating an active material to a mammal on a substantially daily basis, thereby increasing the blood concentration of the active material to an amount effective to control a flea and/or tick infestation in the mammal. Discontinuing administration for at least one day, while maintaining the concentration of the active material at a level that controls the flea and/or tick infestation. After the period of time has elapsed, resuming substantially daily dosing to maintain the blood concentration of the active material in an amount effective to control the flea and/or tick infestation. Also disclosed is a method for establishing a regimen for administering an active material for controlling flea and/or tick infestations in a mammal using a dosage that lower than the label dose for a corresponding time-period.

### RELATED APPLICATIONS

This application is a continuation of International Patent Application No. PCT/US2022/019913, filed Mar. 11, 2022, which claims priority to U.S. Provisional Patent Application No. 63/159,801, filed Mar. 11, 2021, both of which are hereby incorporated herein by reference.

### TECHNICAL FIELD

The teachings of this disclosure generally relate to a method of administering an active material in a feed to control flea and tick infestations in mammals.

### BACKGROUND

In addition to being a nuisance, ticks and fleas create significant health risks to mammals. They are vectors of disease and, when they infest livestock, cause significant economic damage as well.

Ticks are vectors of a number of different pathogenic agents in mammals. Examples of diseases which are caused by ticks include borreliosis (Lyme disease caused by Borrelia burgdorferi), babesiosis (or piroplasmosis caused by Babesia microti) and rickettsiosis (Rocky Mountain spotted fever). Ticks also release toxins, which can cause inflammation or paralysis in the host.

Tick infestations of wild animals such as deer, elk, caribou, moose, etc., can lead to the spread of diseases from herd to herd or from wild animals to domesticated animals (e.g., cattle, cats and dogs) and humans.

Farm animals are also susceptible to various tick infestations, for example, the tick genus Rhipicephalus, especially those of the species microplus (cattle tick), decoloratus and annulatus. Ticks such as Rhipicephalus microplus are particularly difficult to control because they live in pastures where farm animals graze. In addition to cattle, Rhipicephalus spp. and other tick genera may infest and be found on buffalo, horses, donkeys, goats, sheep, deer, pigs, cats and dogs. A heavy tick burden on mammals can decrease production and damage hides as well as transmit diseases such as babesiosis (“cattle fever”) and anaplasmosis caused by protozoan parasites.

In addition to farm animals, ticks also spread disease to companion animals and humans, including, for example, Lyme disease, ascending paralysis and Rocky Mountain spotted fever.

The most common ectoparasites of dogs and cats worldwide are the cat and dog fleas, Ctenocephalides felis felis and Ctenocephalides canis, respectively. Flea-related infestations are among the leading causes of dermatological issues for canines reported to veterinarians. In addition, the cat flea is known to transmit tapeworms in dogs and cats and has been implicated in the transmission of cat scratch disease and murine typhus as well.

Of particular concern, the health-related risks of tick and flea infestations in companion animals extend to humans. [Center for Disease Control and Prevention, Illnesses on the Rise, Vital Signs, May, 2018, available at https://www.cdc.gov/vitalsigns/vector-borne/] Companion animals such as dogs and cats are increasingly common in households worldwide. Globally, around 471 million dogs and 373 million cats are kept as household pets. In the U.S. alone, pet ownership has tripled in the U.S. since the 1970s. Unfortunately, fleas and ticks can infest these companion animals. Infested companion animals expose their human owners to increased risk of illness. One way to control human risk from fleas and ticks is to control the risk of infestation in companion animals.

Treatments currently available for controlling flea and tick infestations achieve varying degrees of success. Many treatments involve topical or environmental application of chemicals applied to indoor and outdoor surfaces, as well as to the animal. The chemicals used include a variety of carbamates, organophosphates, pyrethrins and pyrethroids, isoxazolines, certain macrocyclic lactones, insect and tick growth regulators (including chitin synthesis inhibitors, juvenile hormone analogs, and juvenile hormones), nitromethylenes, neonicotinoids, pyridines and pyrazoles or fiproles. These compounds often have toxic side effects that are a problem for both the animal and animal owners. In addition, there is evidence that the use of these chemicals may be ineffective due to insecticide and acaricide resistance and treatment deficiencies.

Topical treatments are a well-known method for controlling flea and tick infestations. While there are numerous ways to deliver these therapeutic agents to the coats and skins of mammals, many of these methods are either ineffective and/or present safety risks to the mammal or user during or after the dispensing activity. More particularly, because a physical connection must be achieved between the applicator tip and the drug delivery device when the applicator tip is installed thereon, there is inherently a risk that the connection will be inadequate, thereby permitting some of the therapeutic agent to leak out of the device and into physical contact with the user. For example, in the case of larger canines, it may be difficult to maneuver the dispenser with one hand and maintain the canine in place with the other hand, resulting in some, if not all, of the substance being spilled on the floor or on the person applying it instead of reaching the canine's skin. Not only is this leakage wasteful and messy, it also places the user at a heightened risk of suffering from a skin irritation or other such health concern, particularly if the user comes into direct contact with the agent.

Oral treatments are also available for companion animals. However, to be effective, the owner must administer a treatment once every 30-90 days, for example. The extended time between treatments creates compliance issues when owners forget to administer doses.

Despite the availability of effective treatments, a recent study by The Harris Poll found that 33% of pet owners do not routinely protect their pets against fleas and ticks at all. Another study found that pet owners purchased, on average, only 4 months of flea and tick prevention products per year per pet, despite being told that pets needed to be given flea and tick prevention treatments year-round. Thus, there continues to be a need for relatively safe, effective agents for controlling flea and tick infestations on companion animals that is easier for owners to remember to use.

Surprisingly, it has been discovered by the inventors that treatment with an active material, including but not limited to spinosyns and isoxazolines, can provide improved control over flea and tick infestations when orally administered in smaller, more frequent/chronic doses. The administration is discussed below as being combined with feed. However, it is also contemplated that the active material may be administered by itself or in a dosage form other than feed, such as a chew, tablet, liquid, gel or other suitable form for oral administration. Advantageously, by using smaller, more frequent doses, less total active material is required over the same time period to control flea and tick infestations. For example, if 30 mg/kg of isoxazoline would be needed for a single dose in a 30-day (1-month) period, as little as 0.16-0.83 mg/kg per day, or 5-25 mg/kg cumulative over the same 30-day period, may be needed with the change to smaller and more frequent doses.

Advantageously, the total amount of active material, e.g., isoxazoline and/or spinosyn, required for a therapeutically effective once-monthly dose can be reduced by 10-87.5% by converting to daily administration. However, from a practical perspective, at least two problems arise: (1) creating a homogenous feed; and (2) analytical control testing for a very small dose of active material may be difficult to accomplish. The analytical matrix from feeds can be quite complex and difficult to assay. Assays will be in the parts per million to billion range for some needed dose and feed concentrations. Thus, it is possible that one of skill in the art may opt to increase the daily dose such that the total of the daily doses over the course of one month equals the prior art once-monthly dose or is even higher, for example, 200% of the prior art once-monthly dose. This may be done to help ensure homogeneity as well as increase assay accuracy and decrease analytical variability when administering the dose as part of a daily feed.

Further, until the inventor's remarkable discovery, spinosyns were generally considered ineffective for tick control because the doses were administered on a monthly basis and the amount of spinosyn in the animal's blood drops too quickly to control tick infestations.

The method and composition taught herein have the further advantage of encouraging compliance because the smaller doses of an active ingredient can be incorporated into a feed. Since owners naturally follow a daily feeding regimen in any event, this makes it less likely that owners will forget or neglect to administer the treatment. Thus, this disclosure provides a method for prolonged control of ticks in a safer and more effective manner than that achieved with previously known treatment methodologies. All the owner need remember is to feed their pet as they normally would.

Further, the bioavailability of certain active materials can be improved by administering them with feed. Thus, this disclosure provides a method for prolonged control of fleas and ticks in a safer and more effective manner than that achieved with previously known treatments.

The terms “active material” and “active ingredient” are used interchangeably herein and refer to a biologically, nutritionally or pharmaceutically active substance for controlling a flea and/or tick infestation that is delivered to a mammal via a dosage form such as a tablet, a capsule, a liquid, a gel, a medicated feed, a treat, a chew, etc.

Spinosyns are one possible active material. Spinosyns are naturally derived fermentation products. They are macrolides produced by cultivation of Saccharopolyspora spinosa. The fermentation of S. spinosa produces many factors, including spinosyn A and spinosyn D (also called A83543A and A8354D). Spinosyn A and spinosyn D are the two spinosyns that are most active as insecticides. A product comprised mainly of these two spinosyns is available commercially under the generic name “spinosad.” The major spinosyn factor, spinosyn A, is particularly known to have an excellent human and mammal safety and toxicological profile.

Each spinosyn has a 12-membered macrocyclic ring that is part of an unusual tetracyclic ring system to which two different sugars are attached, the amino-sugar forosamine and the neutral sugar 2N,3N,4N-(tri-O-methyl)rhamnose. This unique structure sets the spinosyns apart from other macrocyclic compounds.

Spinosyn A was the first spinosyn isolated and identified from the fermentation broth of Saccharopolyspora spinosa. Subsequent examination of the fermentation broth revealed that S. spinosa produced a number of spinosyns that have been called spinosyns A to J (A83543A to J). The primary components are spinosyns A and D. Additional spinosyns, lettered from K to W, have been identified from mutant strains of S. spinosa. The various spinosyns are characterized by differences in the substitution patterns on the amino group of the forosamine, at selected sites on the tetracyclic ring system and on the 2N,3N,4N-(tri-O-methyl)rhamnose group.

Boeck et al. described spinosyns A-H and J (which they called A83543 factors A, B, C, D, E, F, G, H and J), and salts thereof, in U.S. Pat. No. 5,362,634 (issued Nov. 8, 1994); U.S. Pat. No. 5,496,932 (issued Mar. 5, 1996); and U.S. Pat. No. 5,571,901 (issued Nov. 5, 1996). Mynderse et al. described spinosyns L-N (which they called A83543 factors L, M and N), their N-demethyl derivatives, and salts thereof, in U.S. Pat. No. 5,202,242 (issued Apr. 13, 1993); and Turner et al. described spinosyns Q-T (which they called A83543 factors Q, R, S and T), their N-demethyl derivatives, and salts thereof, in U.S. Pat. No. 5,591,606 (issued Jan. 7, 1997) and U.S. Pat. No. 5,631,155 (issued May 29, 1997). Spinosyns K, O, P, U, V, W and Y are described, for example, by Carl V. DeAmicis, James E. Dripps, Chris J. Hatton and Laura I. Karr in American Chemical Society's Symposium Series: Phytochemicals for Pest Control, Chapter 11, “Physical and Biological Properties of Spinosyns: Novel Macrolide Pest-Control Agents from Fermentation,” pages 146-154 (1997).

The spinosyns can react to form salts that are also useful in the methods and formulations of this disclosure. The salts are prepared using standard procedures for salt preparation. For example, spinosyn A can be neutralized with an appropriate acid to form an acid addition salt. The acid addition salts of spinosyns are particularly useful. Representative suitable acid addition salts include salts formed by reaction with either an organic or inorganic acid such as, for example, sulfuric, hydrochloric, phosphoric, acetic, succinic, citric, lactic, maleic, fumaric, cholic, pamoic, mucic, glutamic, camphoric, glutaric, glycolic, phthalic, tartaric, formic, lauric, stearic, salicylic, methanesulfonic, benzenesulfonic, sorbic, picric, benzoic, cinnamic and like acids.

The term “spinosyn” as used herein refers to an individual spinosyn factor (spinosyn A, B, C, D, E, F, G, H, J, K, L, M, N, O, P, Q, R, S, T, U, V, W or Y), an N-demethyl derivative of an individual spinosyn factor, a chemically modified spinosyn such as spinetoram, a salt of any of the aforementioned, a metabolite of any of the aforementioned, a physiologically acceptable derivative thereof, or a combination thereof.

Spinosyns also provide advantages because they are very effective against fleas and/or ticks with post-treatment residual protection, when the dosages described herein are used according to the method disclosed herein. Furthermore, spinosyns have no insecticidal or acaricidal cross-resistance to existing compounds. Thus, they are especially useful against flea and/or tick populations on mammals that have existing levels of resistance to currently used products. Spinosyns, therefore, can be used in integrated pest management (IPM) programs to extend the lifeline of commonly used products where resistance is not well developed or has not yet developed.

Isoxazolines are another possible active material. Isoxazolines are a class of five-membered heterocyclic chemical compounds, containing one atom each of oxygen and nitrogen which are located adjacent to one another, as depicted below:

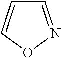

Isoxazolines are all derivatives of isoxazole. They are structural isomers of the more common oxazolines and exist in three different isomers depending on the location of the double bond.

Isoxazoline derivatives are known. For example, WO2007/105814, WO2008/122375, and WO2009/035004 disclose certain alkylene linked amides. WO2010/032437 discloses that the benzyl amide can be moved to the position ortho to the isoxazoline. WO2007/075459 discloses phenyl isoxazolines substituted with 5- to 6-membered heterocycles, and WO2010/084067 and WO2010/025998 disclose phenyl isoxazolines substituted with 10- to 11-membered fused aryl and heteroaryls. Chiral processes for manufacturing isoxazolines are disclosed in WO2011/104089 and WO2009/063910.

A number of isoxazolines compounds are known, including but not limited to 4-(5-methyl-5-substituted pyrrolyl-4,5-dihydroisoxazole-3-yl) benzoic acid amide derivatives; 4-(5-substituted carbamoylmethyl-4,5-dihydroisoxazole-3-yl) benzoic acid amide derivatives; 3-(5-substituted carbamoylmethyl-5-substituted alkyl-4,5-dihydroisoxazole-3-yl) benzoic acid amide derivatives; 4-(5-substituted carbamoylmethyl-4,5-dihydroisoxazole-3-yl) benzamidine derivatives; 4-(5-substituted-5-substituted aryl-4,5-dihydroisoxazole-3-yl)benzoic acid amide compounds; 3-(4-substituted phenyl)-4,5-dihydroisoxazole derivatives; 5-substituted alkyl-3,5-bis substituted phenyl-4,5-dihydroisoxazole derivatives; 3-alkoxyphenyl-5-substituted-5-phenyl-4,5-dihydroisoxazole derivatives; 3-alkoxyphenyl-5-substituted alkyl-5-substituted carbamoyl-4,5-dihydroisoxazole derivatives; 3,4-halophenyl)-5-substituted-5-substituted phenyl-4,5-dihydroisoxazole derivatives; 3-(4-nitrophenyl)-5-substituted-5-substituted phenyl-4,5-dihydroisoxazole derivatives; 4-hydroxyiminomethyl benzoic acid amide derivatives; 4-hydroxyiminomethyl-N,N-dimethyl benzoic acid amide; 4-hydroxyiminomethyl benzoyl piperidine derivatives; 4-hydroxyiminomethyl-N-bicycloalkyl benzoic acid amide derivatives; 6-(hydroxyiminomethyl) pyridine-2-carboxamide derivatives; haloalkenylbenzene derivatives, such as substituted 3,3,3-trifluoro-2-propenylbenzene derivatives; 4-(isoxazolinyl)-benzamides, such as substituted 4-(5-(halomethyl)-5-phenyl-isoxazolin-3-yl)-benzamides; 4-(isoxazolinyl)-benzothioamides, such as substituted 4-(5-(halomethyl)-5-phenyl-isoxazolin-3-yl)-benzothioamides; dihydroisoxazole compounds; and spirocyclic substituted isoxazolines.

Isoxazolines of particular interest for controlling flea and/or tick infestations in mammals are afoxolaner (chemical names: (a) 1-Naphthalenecarboxamide, 4-[5-[3-chloro-5-[(trifluoromethyl)phenyl]-4,5-dihydro-5-(trifluoromethyl)-3-isoxazolyl]-N-[2-oxo-2-[(2,2,2-trifluoroethyl)amino]ethyl]-; or (b) 4-{5-[3-chloro-5-(trifluoromethyl)phenyl]-5-(trifluoromethyl)-4,5-dihydroisoxazol-3-yl}-N-{2-oxo-2-[(2,2,2-trifluoroethyl)amino]ethyl}naphthalene-1-carboxamide), fluralaner (chemical names: (a) Benzamide, 4-[5-(3,5-dichlorophenyl)-4,5-dihydro-5-(trifluoromethyl)-3-isoxazolyl]-2-methyl-N-[2-oxo-2-[(2,2,2-trifluoroethyl)amino]ethyl]-; or (b) 4-[5-(3,5-dichlorophenyl)-5-(trifluoromethyl)-4,5-dihydro-1,2-oxazol-3-yl]-2-methyl-N-{2-oxo-2-[(2,2,2-trifluoroethyl)amino]ethyl}benzamide), sarolaner (chemical names: (a) Ethanone, 1-[5′-[(5S)-5-(3,5-dichloro-4-fluorophenyl)-4,5-dihydro-5-(trifluoromethyl)-3-isoxazolyl]spiro[azetidine-3,1′(3′H)-isobenzofuran]-1-yl]-2-(methylsulfonyl)-; or (b) 1-{5′-[(5S)-5-(3,5-dichloro-4-fluorophenyl)-5-(trifluoromethyl)-4,5-dihydroisoxazol-3-yl]-3′-H-spiro[azetidine-3,1′-[2]benzofuran]-1-yl}-2-(methylsulfonyl)ethanone), lotilaner (chemical names: (a) 2-Thiophenecarboxamide, 5-[(5S)-4,5-dihydro-5-(3,4,5-trichlorophenyl)-5-(trifluoromethyl)-3-isoxazolyl]-3-methyl-N-[2-oxo-2-[(2,2,2-trifluoroethyl)amino]ethyl]-; or (b) 3-methyl-N-{2-oxo-2-[(2,2,2-trifluoroethyl)amino]ethyl }-5-[(5S)-5-(3,4,5-trichlorophenyl)-5-(trifluoromethyl)-4,5-dihydro-1,2-oxazol-3-yl]thiophene-2-carboxamide), esafoxolaner (chemical names: (a) 1-Naphthalenecarboxamide, 4-[(5S)-5-[3-chloro-5-(trifluoromethyl)phenyl]-4,5-dihydro-5-(trifluoromethyl)-3-isoxazolyl]-N-[2-oxo-2-[(2,2,2-trifluoroethyl)amino]ethyl]-; or (b) (S)-4-(5-(3-chloro-5-(trifluoromethyl)phenyl)-5-(trifluoromethyl)-4,5-dihydroisoxazol-3-yl)-N-(2-oxo-2-((2,2,2-trifluoroethyl)amino)ethyl)-1-naphthamide), tigolaner (chemical names: (a) Benzamide, 2-chloro-N-(1-cyanocyclopropyl)-5-[1′-methyl-3′-(1,1,2,2,2-pentafluoroethyl)-4′-(trifluoromethyl)[1,5′-bi-1H-pyrazol]-4-yl]-; or (b) 2-chloro-N-(1-cyanocyclopropyl)-5-[2′-methyl-5′-(pentafluoroethyl)-4′-(trifluoromethyl)-2′H-[1,3′-bipyrazol]-4-yl]benzamide), umifoxolaner (chemical names: (a) 1-Naphthalenecarboxamide, 4-[(5S)-5-[3-chloro-4-fluoro-5-(trifluoromethyl)phenyl]-4,5-dihydro-5-(trifluoromethyl)-3-isoxazolyl]-N-[2-oxo-2-[(2,2,2-trifluoroethyl)amino]ethyl]-; or (b) 4-{(5S)-5-[3-chloro-4-fluoro-5-(trifluoromethyl)phenyl]-5-(trifluoromethyl)-4,5-dihydroisoxazol-3-yl}-N-{2-oxo-2-[(2,2,2-trifluoroethyl)amino]ethyl}naphthalene-1-carboxamide), modoflaner (chemical name: 6-fluoro-N-(2-fluoro-3-{[4-(heptafluoropropan-2-yl)-2-iodo-6-(trifluoromethyl)phenyl]carbamoyl}phenyl)pyridine-3-carboxamide), and mivorilaner (chemical names: (a) 4H-Cyclopenta[c]thiophene-1-carboxamide, 3-[(5S)-5-(3,5-dichloro-4-fluorophenyl)-4, 5-dihydro-5-(trifluoromethyl-3-isoxazolyl]-N-[2-[(2,2-difluoroethyl)amino]-2-oxoethyl]-5,6-dihydro-); or (b) 3-[(5S)-5-(3,5-dichloro-4-fluorophenyl)-5-(trifluoromethyl)-4,5-dihydroisoxazol-3-yl]-N-[2-[(2, 2-difluoroethyl)amino]-2-oxoethyl]-5, 6-dihydro-4H-cyclopenta[c]thiophene-1-carboxamide.

More particularly, isoxazolines with the following structures are suitable for the methods and formulations of this disclosure:

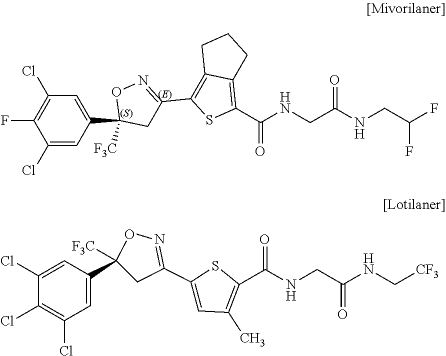

Some isoxazolines can react to form salts that are also useful in the methods and formulations of this disclosure. The salts may be prepared using standard procedures for salt preparation. For example, suitable salts can be acid addition salts such as hydrohalogenated acids, e.g., hydrofluoric acid, hydrochloric acid, hydrobromic acid and hydroiodide, nitric acid, sulfuric acid, phosphoric acid, chloric acid, perchloric acid, salts of sulfonic acids, e.g., methanesulfonic acid, ethanesulfonic acid, trifluoromethanesulfonic acid, benzenesulfonic acid, p-toluenesulfonic acid, salts of carboxylic acids, e.g., valeric acids, formic acid, acetic acid, propionic acid, trifluoroacetic acid, fumaric acid, tartaric acid, oxalic acid, maleic acid, malic acid, succinic acid, benzoic acid, mandelic acid, ascorbic acid, lactic acid, gluconic acid, citric acid or salts of amino acids, e.g., glutamic acid and aspartic acid. Alternatively, metal salts are also suitable for the present disclosure. For example, alkali metal salts, e.g., lithium, sodium and potassium, and alkaline earth metals, e.g., calcium, barium and magnesium, or salts of aluminum.

The terms “isoxazoline” and “isoxazoline or a derivative thereof” as used herein refer to any isoxazoline, isoxazoline derivative, a salt thereof, a metabolite thereof, or a combination thereof.

Isoxazolines also provide advantages because they are very effective against fleas and ticks with post-treatment residual protection when orally administered in smaller, more frequent/chronic doses. Furthermore, isoxazolines have no known insecticidal or acaricidal cross-resistance to existing compounds. Thus, they are especially useful against flea and tick populations on mammals that have existing levels of resistance to currently used products. Isoxazolines, therefore, can be used in integrated pest management (IPM) programs to extend the lifeline of commonly used products where resistance is not well developed or has not yet developed.

Systemic efficacy (e.g., ingestion of blood containing an active material such as spinosyns or isoxazoline by fleas and ticks) provides a different mode of exposure compared to topically applied formulations where contact with the flea or tick at the skin surface is the mode of exposure. The advantages of oral systemic treatments and killing of fleas and ticks from their ingestion of blood, compared to topical applications and contact killing, include:
a) reduced exposure to the human applicator and children and objects in the mammal's environment (e.g., flooring, carpets, furniture);
b) no worry about loss from exposure of the mammal to water (lakes, streams, bathing, etc.) or from loss due to rubbing;
c) no concern about UV exposure and degradation;
d) no problems with oxidation from oils on skin, etc.; and
e) assurance that the entire dose is administered (compared to a topical application where some of the dose may drip off, rub off and/or remain in the dispensing tube immediately after treatment).

The formulations, or feeds, and methods of this disclosure may further include, in combination with the primary active material, one or more other active substances having therapeutic efficacy. Such active substances include agents efficacious against fleas and ticks. Active substances may include, for example, spinosyns, isoxazolines, avermectins, milbemycins, insect or tick growth regulators (including chitin synthesis inhibitors, juvenile hormone analogs, and juvenile hormones), nitromethylenes, neonicotinoids, pyridines and pyrazoles or fiproles.

The methods of this disclosure are carried out by administering the active material to the mammal in small, frequent doses. To facilitate routine dosing, the administration may be carried out using a daily feed, snack, treat, chew, or other supplemental feed. A number of different feeds are envisioned, provided the manufacturing process(es) and feed compositions do not have deleterious effects related to efficacy, stability and safety on the active material and, if applicable, other active substances. For example, feeds, snacks, treats, or other supplemental feeds in the broad categories of dry, semi-moist, canned-retorted feeds or fresh refrigerated feeds may be adapted for use with this disclosure. The mammal receives a maintenance quantity of active material by consuming the feed product on a weekly, semi-weekly or daily basis.

By incorporating smaller doses of active material into an animal feed, snack, treat, chew, or other supplemental feed composition and administering it at an effective frequency (most preferably daily), the blood level of active material rises over time until it reaches an optimal steady state where it can be maintained by a daily or substantially daily dosage. By contrast, when active materials, such as spinosyn or isoxazoline, are orally administered in larger doses at lower frequency, e.g., a single treatment of a large dose that is administered via “treat” once in a 30-day period, the level of active material in the blood spikes at the time of the dose and then declines until the next dose is administered. The administration of a large dose at low frequency means that the animal must consume more active material in each dose so that the blood level of active material does not fall below the necessary level for effective protection before the next dose. Further, because of the rapid and precipitous decline in the blood level of spinosyn as an active material, it has not been possible to maintain a sufficient blood level to control tick infestations using a monthly dosing strategy. In contrast, it is possible to control ticks using spinosyns in the method of this disclosure.

### BRIEF DESCRIPTION OF THE FIGURES

FIG. 1. A graph of the Arithmetic Mean Efficacy, Percent Efficacy=[AM ticks CONTROL−AM ticks TREATED]/AM ticks CONTROL, measured at 14, 21, 30, 37, 44, 51, and 58 days after treatment with spinosad.

FIG. 2. A graph of the Average Plasma Levels of Spinosad in units of ng of spinosad per ml of plasma measured over time (in units of hours after dosing). Values determined for animals treated with either 2.5-5.0 mg of spinosad/kg of animal body mass/day (circles) or a once monthly dose (squares). For animals given a daily dose of spinosad, the dose of spinosad was increased from 2.5 mg/kg/day to 5.0 mg/kg/day of spinosad at about 700 hours.

FIG. 3. A graph of the arithmetic mean efficacy for treatment groups 2-4.

Percent efficacy=[AM fleas CONTROL−AM fleas TREATED]/AM fleas CONTROL

Efficacy measured at 2, 7, 14, 21, 30, and 37 days after Initial Treatment. Group 2 (stippled), Group 3 (right hash), and Group 4 (left hash).

FIG. 4. A graph of the Average Plasma Concentration of Spinosad (ng/ml) versus time in hours. Measured for animals treated with one of the following: 0.25 mg of spinosad/kg of body mass (squares); 0.5 mg of spinosad/kg of body mass (triangles); and 1.0 mg of spinosad/kg of body mass (circles). The last dose was administered at hour 696.

FIG. 5. A graph of the Average Plasma Levels of Spinosad (ng/ml) versus time in hours. Determined for 4 different doses of spinosyn: 0.25 mg/kg (squares); 0.5 mg/kg (triangles); 1.0 mg/kg (circles) and a single monthly dose (diamonds).

FIG. 6. A graph of the arithmetic mean efficacy for treatment Group 2 (stars) and Group 3 (left hash).

Percent efficacy=[AM fleas CONTROL−AM fleas TREATED]/AM fleas CONTROL

Efficacy measured at 2, 7, 14, 21, 30, and 37 days after Initial Treatment.

FIG. 7. A graph of the Arithmetic Mean Efficacy.

Percent Efficacy=[AM ticks CONTROL−AM ticks TREATED]/AM ticks Control

Efficacy is plotted versus time in days. Data collected at 2, 7, 14, 21, 30 and 37 days after the initial dose from canines treated with 0.75 mg of Mivorilaner/kg of animal body mass/day.

FIG. 8. A graph of mivorilaner levels measured in the plasma of treated animals (ng of mivorilaner/mL of plasma) versus time in days after initial treatment (3, 7, 14, 21, 30, and 37 days). Data collected for test subjects: MC3840 (squares), MC0483 (diamonds), MC3500 (stars), MC7440 (triangles), MC7322 (closed circles), and MC9624 (open circles).

FIG. 9. A graph of the Arithmetic Mean Efficacy.

Percent Efficacy=[AM ticks CONTROL−AM ticks TREATED]/AM ticks Control

Efficacy is plotted versus time in days. Data collected at 2, 7, 14, 21, 30, 37 and 44 days after the initial dose from: Group 2, 0.125 mg of mivorilaner/kg of animal body mass/day (stippled); Group 3, 0.25 mg of mivorilaner/kg of animal body mass/day (right hash); Group 4, 0.5 mg of mivorilaner/kg of animal body mass/day (left hash); and Group 5, 1.0 mg of mivorilaner/kg of animal body mass/day (horizontal hash).

FIG. 10. A graph of mivorilaner levels measured in the plasma of treated animals (ng of mivorilaner/mL of plasma) versus time in hours after initial treatment. Data collected from Groups 2-5 as follows: Group 2, 0.125 mg of mivorilaner/kg of animal body mass/day (circles); Group 3, 0.25 mg of mivorilaner/kg of animal body mass/day (squares); Group 4, 0.5 mg of mivorilaner/kg of animal body mass/day (triangles); and Group 5, 1.0 mg of mivorilaner/kg of animal body mass/day (diamonds).

FIG. 11a. Graph of level of mivorilaner in the plasma of the individual adult canines in Group 1. Canines were treated with 0.25 mg of mivorilaner/kg body weight of the canine administered by IV. Plasma concentration expressed in units of ng of mivorilaner/mL plasma versus time in days for specific canines.

FIG. 11b. Graph of level of mivorilaner in the plasma of the individual juvenile canines in Group 2. Canines were treated with 0.25 mg of mivorilaner/kg body weight of the canine administered by IV. Plasma concentration expressed in units of ng of mivorilaner/mL plasma versus time in days for specific canines.

FIG. 11c. Graph of level of mivorilaner in the plasma of the individual adult canines in Group 3. Canines were treated with 1.0 mg of mivorilaner/kg body weight of the canine administered orally. Plasma concentration expressed in units of ng of mivorilaner/mL plasma versus time in days for specific canines.

FIG. 11d. Graph of level of mivorilaner in the plasma of the individual adult canines in Group 4. Canines were treated with 1.0 mg of mivorilaner/kg body weight of the canine administered orally. Plasma concentration expressed in units of ng of mivorilaner/mL plasma versus time in days for specific canines.

FIG. 12. A graph of the Arithmetic Mean Efficacy.

Percent Efficacy=[AM ticks CONTROL−AM ticks TREATED]/AM ticks Control

Efficacy is plotted versus time in days. Data collected from Groups 2-5: 0.05 mg of Afoxolaner/kg of animal body mass/day (Group 2, stars); 0.15 mg of Afoxolaner/kg of animal body mass/day (Group 3, right hash); 0.083 mg of Fluralaner/kg of animal body mass/day (Group 4, left hash); and 0.25 mg of Fluralaner/kg of animal body mass/day (Group 5, horizontal hash). Comb counts conducted at 2, 7, 14, 21 and 30 days after the initial treatment.

FIG. 13. Graph of Mean (SD) Mivorilaner in ng/mL versus time (days) after a single oral dose of mivorilaner. Plasma levels measured in animals dosed with 1 mg of mivorilaner/kg of body mass with five different formulations of kibble (formulations 1, 2, 3, 4, and 5).

FIG. 14. A graph of the Arithmetic Mean Efficacy.

Percent Efficacy=[AM ticks CONTROL−AM ticks TREATED]/AM ticks Control

Efficacy is plotted versus time in days. Data collected from Groups 2-3: 1.0 mg of Mivorilaner/kg of animal body mass/day (Group 2, stars); 1.0 mg of Mivorilaner/kg of animal body mass/day (Group 3, right hash) Comb counts conducted at 3, 8, 15, 30, and 58 days after treatment with mivorilaner.

FIG. 15. Graph of blood levels of mivorilaner in canines fed a feed including different levels of the isoxazoline: 106 mg of isoxazoline/kg of feed (Group 2-square symbol); 106 mg of isoxazoline/kg of feed (Group 3-circle symbol); and 318 mg of isoxazoline/kg of feed (Group 4-triangle symbol).

FIG. 16. A graph of the Arithmetic Mean Efficacy.

Percent Efficacy=[AM fleas CONTROL−AM fleas TREATED]/AM fleas Control

Efficacy is plotted versus time in Days. Data collected from animals treated with 0.75 mg of Mivorilaner/kg of animal body mass/day, data collected at 4, 7, 14, 21, and 37 days after the first dosing.

FIG. 17. A graph of mivorilaner levels measured in the plasma of treated animals (ng of mivorilaner/mL of plasma) versus time in days (3, 7, 14, 21, 30, and 37 days). Data collected from the following test animals: MC3840 (squares), MC0483 (diamonds), MC3500 (*); MC7440 (triangles); MC7322 (closed circles), and MC9624 (open circles).

FIG. 18. A graph of the Arithmetic Mean Efficacy.

Percent Efficacy=[AM fleas CONTROL−AM fleas TREATED]/AM fleas Control

Efficacy is plotted versus time in Days. Data collected from animals treated with various amounts of mivorilaner: Group 2, 0.125 mg of mivorilaner/kg of animal body mass/day (stippled); Group 3, 0.25 mg of mivorilaner/kg of animal body mass/day (right hash); Group 4, 0.5 mg of mivorilaner/kg of animal body mass/day (left hash); and Group 5, 1.0 mg of mivorilaner/kg of animal body mass/day (horizontal hash). The data collected at 2, 7, 14, 21, 30, 37, and 44 days after initial treatment.

FIG. 19. A graph of mivorilaner levels measured in the plasma of treated animals (ng of mivorilaner/mL of plasma) versus time in hours after initial treatment. Data collected from Groups 2-5 as follows: Group 2, 0.125 mg of mivorilaner/kg of animal body mass/day (circles); Group 3, 0.25 mg of mivorilaner/kg of animal body mass/day (squares); Group 4, 0.5 mg of mivorilaner/kg of animal body mass/day (triangles); and Group 5, 1.0 mg of mivorilaner/kg of animal body mass/day (diamonds).

FIG. 20. A graph of the Arithmetic Mean Efficacy.

Percent Efficacy=[AM fleas CONTROL−AM fleas TREATED]/AM fleas Control

Efficacy is plotted versus time in days. Data collected from Groups 2-5: 0.05 mg of Afoxolaner/kg of animal body mass/day (Group 2, stars); 0.15 mg of Afoxolaner/kg of animal body mass/day (Group 3, right hash); 0.083 mg of Fluralaner/kg of animal body mass/day (Group 4, left hash); and 0.25 mg of Fluralaner/kg of animal body mass/day (Group 5, horizontal hash). Samples drawn at 2, 7, and 30 days after the initial treatment.

FIG. 21. A graph of the Arithmetic Mean Efficacy.

Percent Efficacy=[AM ticks CONTROL−AM ticks TREATED]/AM ticks Control

Efficacy is plotted versus time in days. Data collected from canines treated with: 0.26 mg of Lotilaner/kg of animal body mass/day (Group 2, stars); 0.6 mg of Lotilaner/kg of animal body mass/day (Group 3, right hash); 0.016 mg of Sarolaner/kg of animal body mass/day (Group 4, left hash); and 0.036 mg of Sarolaner/kg of animal body mass/day (Group 5, horizontal hash). Comb counts conducted 2, 7, 14, 21, 30, and 35 days after initial treatment with either lotilaner or sarolaner.

FIG. 22. A graph of the Arithmetic Mean Efficacy.

Percent Efficacy=[AM fleas CONTROL−AM fleas TREATED]/AM fleas Control

Efficacy is plotted versus time in days. Data collected from canines treated with: 0.26 mg of Lotilaner/kg of animal body mass/day (Group 2, stars); 0.6 mg of Lotilaner/kg of animal body mass/day (Group 3, right hash); 0.016 mg of Sarolaner/kg of animal body mass/day (Group 4, left hash); and 0.036 mg of Sarolaner/kg of animal body mass/day (Group 5, horizontal hash). Samples drawn at 2, 7, and 35 days after the initial treatment.

### DESCRIPTION

All ratios, percentages, and parts discussed herein are “by weight” unless otherwise specified.

The term “controlling a tick infestation” refers to preventing, treating, minimizing or eliminating an infestation by ticks on a mammal.

The term “tick” refers to any member of the order Ixodida. The term “tick” includes the egg, larval, nymph, and adult stages of development. More particularly, the term tick includes ticks of the families Ixodidae and Argasidae. More particularly, the term “tick” includes species of the genera Africaniella, Amblyomma, Anomalohimalaya, Bothriocroton, Dermacentor, Haemaphysalis, Hyalomma, Ixodes, Margaropus, Nosomma, Rhipicentor, Rhipicephalus, Antricola, Argas, Nothoaspis, Ornithodoros, and Otobius.

The term “controlling a flea infestation” refers to preventing, treating, minimizing or eliminating an infestation by fleas on a mammal.

The term “flea” refers to any member of the order Siphonaptera. The term “flea” includes the egg, larval, pupal, and adult stages of development.

The term “mammal” refers to any member of the class Mammalia. In particular, it may refer to wild mammals, such as wolves, coyotes, jackals, deer, elk, moose, reindeer, and the like. It may also refer to farm animals, such as cows, sheep, pigs, bison, horses and the like. It may also refer to companion animals. It may also refer to humans.

The term “companion animal” refers to any domestic animal that may be kept as a pet. This includes, but is not limited to, horses, dogs, wolves, coyotes, cats, hamsters, gerbils, mice, guinea pigs, ferrets, rabbits, etc.

The term “canine” refers to any member of the genus Canis, which includes such species as wolves, dogs, coyotes and jackals.

The term “feline” refers to any member of the subfamily Felidae, which includes such species as the domestic cat, bobcats, wildcats, ocelots, members of the genus lynx, Pallas's cat and cougars.

In carrying out the methods of this disclosure, a “feed” is an animal feed or treat, snack or other supplemental feed that may be administered daily or substantially daily. By using different forms of feed, e.g., kibble and treats, a pet owner may vary the canine's meals and snacks from time to time while still conveniently administering a daily dose of spinosyn.

The term “chew” refers to a treat that has flavor and aromatic properties that are appealing to a canine, but typically has no nutritional value. In carrying out the methods of this disclosure, a “feed” and/or a “chew” may be used interchangeably.

The term “effective time,” also referred to herein as “effective duration,” for the purposes of this disclosure includes at least the duration of administration needed to bring the level of active material in the mammal's blood to a sufficiently high level for controlling fleas and/or ticks, i.e., a “therapeutically effective” level. In some embodiments, the effective time may be as little as three days. In other instances, the effective time may be seven days or fifteen days or longer. As discussed below, the effective time will vary based on how frequently the active material is administered.

As just alluded, the “effective time” will vary as a function of the frequency at which the active material is administered. The term “effective frequency” as used herein means the number of doses over a given time that produce a therapeutically effective concentration of active material in the mammal's blood. In all events, the term “effective frequency” as used herein contemplates multiple doses of the active material per month. One of skill in the art will appreciate that the active material may be administered in a range of frequencies. For example, the active material may be administered at a frequency of daily, every other day, every third day, once per week or even at inconsistent time intervals.

Further, as discussed above, the effective frequency may affect the duration required to obtain a therapeutically effective level of active material in the mammal's blood. By way of example, if the mammal were being fed the feed composition daily, the duration of administration required to achieve a therapeutically effective level of active material in the mammal's blood, and thus the “effective time,” would be comparatively less than if the mammal were being fed the feed composition only once or twice per week.

Further, the effective frequency is influenced by the amount of the daily dose in mg/kg of body weight of the mammal. Particularly, at slightly higher daily doses, missed doses have less of an impact on efficacy.

Further, the effective frequency is influenced by the duration of treatment. In the initial stages, e.g., before the amount of active material in the mammal's blood has reached a therapeutically effective level, the active material may need to be administered more often than would be necessary after a longer period of use, i.e., once a therapeutically effective level is obtained.

For purposes of this disclosure, “substantially daily” means a sufficiently regular basis such that the active material concentration in the mammal's blood rises to and remains at a therapeutically effective level. For example, the disclosed daily feed composition can preferably be fed to a mammal every day indefinitely. However, as a practical matter, there are many reasons why days may be missed or skipped periodically. For example, the mammal may be ill or the owner may run out of the daily medicated feed composition. The disclosed method is robust enough that the mammal will still be protected from fleas and/or ticks to some extent even with occasional interruptions in daily administration of the active material. In carrying out the method of this disclosure, the term “substantially daily” includes at least 10 days per month, more preferably at least 15 days per month, still more preferably at least 20 days per month. All of these feeding frequencies, whether they be, e.g., three times per week, every other day or daily, fit under the umbrella of substantially daily provided that they promote the active ingredient reaching and maintaining a therapeutically effective level of the active ingredient in the mammal's blood.

Surprisingly, it has been found that in some circumstances, depending on the active material and the flea and/or tick whose population is intended to be controlled, the disclosed method is sufficiently robust that the administration of the active ingredient could be interrupted for a period of time and yet the concentration of active material in the mammal's blood will remain sufficiently high that control of the flea and/or tick population is maintained during the time period the feeding has been interrupted. For example the feed with the active ingredient may be interrupted for 1 day, 3 days, 7 days or more than a week. Several illustrative examples demonstrating this are presented below. See Example 1, Example 2, Example 4, Example 7, Example 8, Example 9, Example 10 and Example 12 (demonstrating commercial label effectiveness after administration of the active ingredient was interrupted). This ability to interrupt administration of the active ingredient and still maintain a therapeutically effective blood concentration is present over a wide range of dosage levels, including dosages from about 10% to about 250% of an equivalent daily dose.

With the exception of food animal production, it is very difficult to administer drugs in animal food because of the problem of feed variability and an inability to maintain a therapeutic drug level through the dosing period. For example, some owners feed their dog once a day and some more than once a day. Cats often eat small meals throughout the day. Some animals may not eat for a few days due to illness. As an example, Ivermectin is used extensively in both food producing animal and companion animals, but it has only been used in food producing animals through in-feed administration. However, the inventors have discovered that building the concentration of certain active ingredient in a mammal's blood over time by using smaller, substantially daily doses overcomes the problem of feed variability.

Advantageously, an interruption in the dose administration according to the inventive method is significantly less deleterious in controlling flea and tick infestations compared with an interruption in dose administration for the traditional method, i.e., large doses administered less frequently. Preferably, the interrupting of dose administration does not occur until the blood concentration of active material reaches a steady state or quasi-steady state level sufficiently high to control the flea and/or tick population. On the other hand, it is also possible to interrupt and then resume the daily dosing/feeding after first beginning the dosing/feeding regimen and before the blood concentration reaches the desired amount necessary to control the flea and/or tick population. In this case, it would normally take a longer period of time for the substantially daily dosing/feeding to cause the concentration of active material to reach an amount effective to control the flea and/or tick population of the mammal.

The term “therapeutically effective” means that the dose or blood level of an active material is sufficient to control the flea and/or tick infestation better than if no drug were present. The active material may be present on its own or with one or more additional active substances. Preferably it controls the flea and/or tick infestation at around at least 50% better than if no drug were present, and more preferably it controls the flea and/or tick infestation at about at least 90% better than if no drug were present.

In carrying out the methods of this disclosure, an effective or therapeutically effective amount of an active material is administered orally to the mammal. The term “effective amount” or “therapeutically effective amount” refers to the amount needed to control the flea and/or tick infestation. As those in the art will understand, this amount will vary depending upon a number of factors. These factors include, for example, the type of mammal being treated and its weight and general physical condition.

While this disclosure describes concentrations of spinosyn in terms of feeds such as kibble, it also contemplates administration using other dosage forms, such as treats or chews. It is also contemplated that the spinosyn may be administered by itself or in a tablet, liquid, gel or other suitable form for oral administration. One of skill in the art will appreciate that the concentration of spinosyn will vary according to the particular dosage form. For example, where the dosage form is a treat or chew, the concentration of spinosyn in the treat or chew will be greater than, e.g., the concentration of spinosyn in kibble. For example, if the daily dose of spinosyn based on the weight of the canine is 10 mg, then a typical 5 g treat or chew would contain about 0.2 percent spinosyn (by weight). Since the amount of kibble consumed in a day is more than 5 g, the percent spinosyn in kibble will be smaller.

In general, an effective amount of spinosyn for controlling flea infestations refers to a daily dose of from about 0.125 to about 4.5 mg of the spinosyn/kg of body weight of the mammal. More commonly, the effective amount is from about 0.2 to about 3.75 mg/kg of body weight of the mammal.

Animal feeds for controlling flea infestations will typically contain from about 0.0005 to about 0.2 percent of the spinosyn (by weight) in the feed. Preferably between about 0.001 to about 0.12 percent of the spinosyn (by weight) in the feed. Most preferably between about 0.003 to about 0.06 percent of the spinosyn (by weight) in the feed.

In general, an effective amount of spinosyn for controlling tick infestations refers to a daily dose of from about 0.625 to about 4.5 mg of the spinosyn/kg of body weight of the mammal. More commonly, the effective amount is from about 1 to about 3.75 mg/kg of body weight of the mammal.

Animal feeds for controlling tick infestations will typically contain from about 0.005 to about 2 percent of spinosyn (by weight) in the feed. Preferably between about 0.01 to about 0.5 percent of spinosyn (by weight) in the feed. Most preferably between about 0.03 to about 0.2 percent of spinosyn (by weight) in the feed.

Isoxazolines vary in potency. Thus, the effective amount of isoxazoline must be calculated for each particular isoxazoline used in the method according to this disclosure. In general, the effective amount for a daily dose of an isoxazoline will be in the range of about 12.5%-90% of the approved label dose for said isoxazoline divided by length of the dosing/retreatment interval (e.g., 30 days for a product administered monthly). The particular dose selected may be sufficient to raise the mammal's blood concentration of said isoxazoline to a therapeutically effective level within about 7 days of substantially daily administrations, more preferably within about 5 days of substantially daily administrations, most preferably within about 3 days of substantially daily administrations.

While this disclosure describes concentrations of isoxazoline in terms of feeds such as kibble, it also contemplates administration using other dosage forms, such as treats or chews. It is also contemplated that the isoxazoline may be administered by itself or in a tablet, liquid, gel or other suitable form for oral administration. One of skill in the art will appreciate that the concentration of isoxazoline will vary according to the particular dosage form. For example, where the animal feed is a treat, the concentration of isoxazoline in the treat will be greater than, e.g., the concentration of isoxazoline in a kibble. For example, if the daily dose of isoxazoline based on the weight of the canine is 20 mg, then a typical 5 g treat may contain about 0.004 percent isoxazoline (by weight). Since the amount of kibble consumed in a day is more than 5 g, the percent isoxazoline in kibble will be smaller.

For example, an effective amount of mivorilaner for controlling flea infestation may be a dose of from about 0.04 to about 1.5 mg of mivorilaner/kg of body weight of the mammal. More commonly, the effective amount is from about 0.07 to about 1.25 mg/kg of body weight of the mammal.

Animal feeds for controlling flea infestation will typically contain from about 0.0001 to about 0.08 percent of mivorilaner (by weight) in the feed. Preferably between about 0.0002 to about 0.05 percent of mivorilaner (by weight) in the feed. Most preferably between about 0.0006 to about 0.03 percent of mivorilaner component or components (by weight) in the feed.

In another example, an effective amount of lotilaner for controlling flea infestation may be a dose of from about 0.017 to about 0.6 mg of lotilaner/kg of body weight of the mammal. More commonly, the effective amount is from about 0.027 to about 0.5 mg/kg of body weight of the mammal.

Animal feeds for controlling flea infestation will typically contain from about 0.00004 to about 0.03 percent of lotilaner (by weight) in the feed. Preferably between about 0.00008 to about 0.02 percent of lotilaner (by weight) in the feed. Most preferably between about 0.0002 to about 0.001 percent of lotilaner component or components (by weight) in the feed.

In another example, an effective amount of afoxolaner for controlling flea infestation may be a dose of from about 0.002 to about 0.075 mg of afoxolaner/kg of body weight of the mammal. More commonly, the effective amount is from about 0.003 to about 0.0625 mg/kg of body weight of the mammal.

Animal feeds for controlling flea infestation will typically contain from about 0.000005 to about 0.03 percent of afoxolaner (by weight) in the feed. Preferably between about 0.00001 to about 0.02 percent of afoxolaner (by weight) in the feed. Most preferably between about 0.00003 to about 0.0012 percent of afoxolaner component or components (by weight) in the feed.

In another example, an effective amount of sarolaner for controlling flea infestation may be a dose of from about 0.001 to about 0.036 mg of sarolaner/kg of body weight of the mammal. More commonly, the effective amount is from about 0.0016 to about 0.03 mg/kg of body weight of the mammal.

Animal feeds for controlling flea infestation will typically contain from about 0.000002 to about 0.03 percent of sarolaner (by weight) in the feed. Preferably between about 0.000004 to about 0.02 percent of sarolaner (by weight) in the feed. Most preferably between about 0.0003 to about 0.0006 percent of sarolaner component or components (by weight) in the feed.

In another example, an effective amount of fluralaner for controlling flea infestation may be a dose of from about 0.008 to about 0.3 mg of fluralaner/kg of body weight of the mammal. More commonly, the effective amount is from about 0.013 to about 0.25 mg/kg of body weight of the mammal.

Animal feeds for controlling flea infestation will typically contain from about 0.00002 to about 0.03 percent of fluralaner (by weight) in the feed. Preferably between about 0.00004 to about 0.02 percent of fluralaner (by weight) in the feed. Most preferably between about 0.0001 to about 0.006 percent of fluralaner component or components (by weight) in the feed.

In another example, an effective amount of umifoxolaner for controlling flea infestation may be a dose of from about 0.001 to about 0.04 mg of umifoxolaner/kg of body weight of the mammal. More commonly, the effective amount is from about 0.0017 to about 0.03125 mg/kg of body weight of the mammal.

Animal feeds for controlling flea infestation will typically contain from about 0.000002 to about 0.03 percent of umifoxolaner (by weight) in the feed. Preferably between about 0.000005 to about 0.02 percent of umifoxolaner (by weight) in the feed. Most preferably between about 0.00001 to about 0.0006 percent of umifoxolaner component or components (by weight) in the feed.

In another example, an effective amount of esafoxolaner for controlling flea infestation may be a dose of from about 0.001 to about 0.038 mg of esafoxolaner/kg of body weight of the mammal. More commonly, the effective amount is from about 0.0017 to about 0.03125 mg/kg of body weight of the mammal.

Animal feeds for controlling flea infestation will typically contain from about 0.000002 to about 0.03 percent of esafoxolaner (by weight) in the feed. Preferably between about 0.000005 to about 0.02 percent of esafoxolaner (by weight) in the feed. Most preferably between about 0.00001 to about 0.0006 percent of esafoxolaner component or components (by weight) in the feed.

In another example, an effective amount of tigolaner for controlling flea infestation may be a dose of from about 0.001 to about 0.038 mg of tigolaner/kg of body weight of the mammal. More commonly, the effective amount is from about 0.0017 to about 0.03125 mg/kg of body weight of the mammal.

Animal feeds for controlling flea infestation will typically contain from about 0.000002 to about 0.03 percent of tigolaner (by weight) in the feed. Preferably between about 0.000005 to about 0.02 percent of tigolaner (by weight) in the feed. Most preferably between about 0.00001 to about 0.0006 percent of tigolaner component or components (by weight) in the feed.

For example, an effective amount of mivorilaner for controlling a tick infestation may be a dose of from about 0.21 to about 1.5 mg of mivorilaner/kg of body weight of the mammal. More commonly, the effective amount is from about 0.33 to about 1.25 mg/kg of body weight of the mammal.

Animal feeds for controlling a tick infestation will typically contain from about 0.001 to about 0.4 percent of mivorilaner (by weight) in the feed. Preferably between about 0.002 to about 0.24 percent of mivorilaner (by weight) in the feed. Most preferably between about 0.003 to about 0.12 percent of mivorilaner component or components (by weight) in the feed.

In another example, an effective amount of lotilaner for controlling a tick infestation may be a dose of from about 0.083 to about 0.6 mg of lotilaner/kg of body weight of the mammal. More commonly, the effective amount is from about 0.133 to about 0.5 mg/kg of body weight of the mammal.

Animal feeds for controlling a tick infestation will typically contain from about 0.0004 to about 0.16 percent of lotilaner (by weight) in the feed. Preferably between about 0.0008 to about 0.1 percent of lotilaner (by weight) in the feed. Most preferably between about 0.002 to about 0.005 percent of lotilaner component or components (by weight) in the feed.

In another example, an effective amount of afoxolaner for controlling a tick infestation may be a dose of from about 0.01 to about 0.075 mg of afoxolaner/kg of body weight of the mammal. More commonly, the effective amount is from about 0.017 to about 0.0625 mg/kg of body weight of the mammal.

Animal feeds for controlling a tick infestation will typically contain from about 0.00005 to about 0.16 percent of afoxolaner (by weight) in the feed. Preferably between about 0.0001 to about 0.1 percent of afoxolaner (by weight) in the feed. Most preferably between about 0.0003 to about 0.006 percent of afoxolaner component or components (by weight) in the feed.

In another example, an effective amount of esafoxolaner for controlling a tick infestation may be a dose of from about 0.005 to about 0.375 mg of esafoxolaner/kg of body weight of the mammal. More commonly, the effective amount is from about 0.008 to about 0.03125 mg/kg of body weight of the mammal.

Animal feeds for controlling a tick infestation will typically contain from about 0.00002 to about 0.16 percent of esafoxolaner (by weight) in the feed. Preferably between about 0.00005 to about 0.1 percent of esafoxolaner (by weight) in the feed. Most preferably between about 0.0001 to about 0.003 percent of esafoxolaner component or components (by weight) in the feed.

In another example, an effective amount of sarolaner for controlling a tick infestation may be a dose of from about 0.005 to about 0.036 mg of sarolaner/kg of body weight of the mammal. More commonly, the effective amount is from about 0.008 to about 0.03 mg/kg of body weight of the mammal.

Animal feeds for controlling a tick infestation will typically contain from about 0.00002 to about 0.16 percent of sarolaner (by weight) in the feed. Preferably between about 0.00004 to about 0.1 percent of sarolaner (by weight) in the feed. Most preferably between about 0.0001 to about 0.003 percent of sarolaner component or components (by weight) in the feed.

In another example, an effective amount of fluralaner for controlling a tick infestation may be a dose of from about 0.0417 to about 0.3 mg of fluralaner/kg of body weight of the mammal. More commonly, the effective amount is from about 0.067 to about 0.25 mg/kg of body weight of the mammal.

Animal feeds for controlling a tick infestation will typically contain from about 0.0002 to about 0.16 percent of fluralaner (by weight) in the feed. Preferably between about 0.0004 to about 0.1 percent of fluralaner (by weight) in the feed. Most preferably between about 0.001 to about 0.03 percent of fluralaner component or components (by weight) in the feed.

In another example, an effective amount of umifoxolaner for controlling a tick infestation may be a dose of from about 0.005 to about 0.375 mg of umifoxolaner/kg of body weight of the mammal. More commonly, the effective amount is from about 0.008 to about 0.03125 mg/kg of body weight of the mammal.

Animal feeds for controlling a tick infestation will typically contain from about 0.00002 to about 0.16 percent of umifoxolaner (by weight) in the feed. Preferably between about 0.00005 to about 0.1 percent of umifoxolaner (by weight) in the feed. Most preferably between about 0.0001 to about 0.003 percent of umifoxolaner component or components (by weight) in the feed.

In another example, an effective amount of tigolaner for controlling a tick infestation may be a dose of from about 0.005 to about 0.0375 mg of tigolaner/kg of body weight of the mammal. More commonly, the effective amount is from about 0.008 to about 0.03125 mg/kg of body weight of the mammal.

Animal feeds for controlling a tick infestation will typically contain from about 0.00002 to about 0.16 percent of tigolaner (by weight) in the feed. Preferably between about 0.00005 to about 0.1 percent of tigolaner (by weight) in the feed. Most preferably between about 0.0001 to about 0.003 percent of tigolaner component or components (by weight) in the feed.

In one aspect, this disclosure relates to a method of controlling a flea and/or tick infestation in a mammal by administering a systemically active oral composition including an active material and animal feed at a frequency of at least once per week, more preferably three times per week, most preferably substantially daily.

In another aspect, this disclosure relates to a systemically active oral composition that includes an active material and animal feed or chew.

This disclosure also relates to the use of an active material for the manufacture of an animal feed or chew for controlling a flea and/or tick infestation on a mammal.

This disclosure also relates to a method of controlling a flea and/or tick infestation on a mammal for a prolonged time, comprising orally administering daily or substantially daily doses of an effective amount of an active material to the mammal in a daily feed. A daily feed is a feed which is intended to be administered daily, however which may be administered at other frequencies, as described herein. This method is especially useful for controlling fleas and/or ticks on a mammal for a prolonged time comprising orally administering substantially daily doses of an effective amount of an active material to the mammal.

An aspect of this disclosure is the oral administration of an amount of active material that is, in and of itself, ineffective or sub-optimal for controlling a flea and/or tick infestation in a mammal in a single dose, but over time with repeated administrations, as described herein, results in efficacious control of flea and/or tick infestations. Ineffective or sub-optimal means that a single dosing, as well as several dosings, results in less than a 50% reduction in the flea and/or tick infestation, including no, or substantially no, reduction, as compared to no drug administration at all. This reflects the chronic, rather than acute, administration aspect disclosed herein.

Embodiment 1: A method of controlling a flea and/or tick infestation in a mammal in need thereof, comprising:
orally administering to said mammal an effective amount of an active material;
continuing the oral administration substantially daily over a period of days to thereby increase the mammal's blood concentration of the active material to an amount effective to reduce the flea and/or tick infestation;
after the mammal's blood concentration of the active material reaches the amount effective to reduce the flea or tick infestation, discontinuing the oral administration for a time period of at least one day, wherein the mammal's blood concentration of active material remains effective to control the flea and/or tick infestation over the time period.

Embodiment 2: The method of embodiment 1, wherein the time period is at least three consecutive days, wherein the mammal's blood concentration of active material remains effective to control the flea and/or tick infestation over the time period.

Embodiment 3: The method of embodiment 1, wherein the time period is at least seven consecutive days, wherein the mammal's blood concentration of active material remains effective to control the flea and/or tick infestation over the time period.

Embodiment 4: The method of any of embodiments 1-3, further comprising resuming the substantially daily administration after the time period has elapsed and thereby continuing to maintain the mammal's blood concentration of active material in an amount effective to control the flea and/or tick infestation.

Embodiment 5: The method of embodiment 4, wherein the time period comprises a plurality of time periods each being at least one day and all occurring within 30 days.

Embodiment 6: The method of embodiment 1, wherein the active material is selected from the group consisting of a spinosyn and an isoxazoline.

Embodiment 7: The method of embodiment 6, wherein the active material is a spinosyn.

Embodiment 8: The method of embodiment 7, wherein said spinosyn is spinosad.

Embodiment 9: The method of embodiment 6, wherein the active material is an isoxazoline.

Embodiment 10: The method of embodiment 9, wherein the isoxazoline is selected from the group consisting of mivorilaner, fluralaner, sarolaner, afoxolaner, lotilaner, umifoxolaner, esafoxolaner, tigolaner, modoflaner, and salts thereof.

Embodiment 11: The method of any of embodiments 1-10, wherein the active material is a component of a wet or dry feed.

Embodiment 12: The method of embodiment 11, wherein the feed comprises a dry feed.

Embodiment 13: The method of any of embodiments 1-10, wherein the active material is a component of a chew.

Embodiment 14: A method of establishing a regimen for orally administering a reduced dosage of an active material for controlling flea and/or tick infestations in a mammal, the method comprising:
(a) selecting an active material having a prescribed dosage for oral administration to a mammal for controlling fleas and/or ticks, wherein the prescribed dosage is for a prescribed administration regimen of once every 30 days or once every month;
(b) multiplying the amount of the prescribed dosage by about 0.90 or less to yield a reduced dosage;
(c) converting the reduced dosage into a reduced daily dosage; and
(d) providing instructions to administer the active material at the reduced daily dosage to the mammal substantially daily over the course of several days;
whereby, after the instructions to administer are followed, the mammal's flea and/or tick infestation is controlled to the same or greater extent than when administering the prescribed dosage of active material to the mammal at the once every 30 days or once every month intervals.

Embodiment 15: The method of embodiment 14, wherein step (b) comprises multiplying the amount of the prescribed dosage by between about 0.20 to about 0.90.

Embodiment 16: The method of any of embodiments 14 and 15, wherein step (b) comprises multiplying the amount of the prescribed dosage by no more than about 0.70.

Embodiment 17: The method of any of embodiments 14-16, wherein step (b) comprises multiplying the amount of the prescribed dosage by no more than about 0.50.

Embodiment 18: The method of any of embodiments 14-17, wherein the active material is selected from the group consisting of a spinosyn and an isoxazoline.

Embodiment 19: The method of embodiment 18, wherein the active material is a spinosyn.

Embodiment 20: The method of embodiment 19, wherein said spinosyn is spinosad.

Embodiment 21: The method of embodiment 20, wherein step (b) comprises multiplying the amount of the prescribed dosage by about 12.5% to 90%.

Embodiment 22: The method of embodiment 20, wherein the prescribed dosage controls fleas and not ticks and the reduced daily dosage controls fleas and ticks.

Embodiment 23: The method of embodiment 20, wherein performing steps (a)-(d) results in the maximum concentration in the mammal's blood of the active material reaching less than about 10% of the maximum blood concentration reached when administering the prescribed dosage of active material to the mammal at the once every 30 days or once every month intervals.

Embodiment 24: The method of embodiment 18, wherein the active material is an isoxazoline.

Embodiment 25: The method of embodiment 24, wherein the isoxazoline is selected from the group consisting of mivorilaner, fluralaner, sarolaner, afoxolaner, lotilaner, tigolaner, umifoxolaner, esafoxolaner, modoflaner, and salts thereof.

Embodiment 26: The method of embodiment 25, wherein the active material is mivorilaner.

Embodiment 27: The method of embodiment 26, wherein step (b) comprises multiplying the amount of the prescribed dosage by about 0.125 to about 0.9.

Embodiment 28: The method of embodiment 27, wherein performing steps (a)-(d) results in the maximum concentration in the mammal's blood of the active material reaching less than about 20% of the maximum blood concentration reached when administering the prescribed dosage of active material to the mammal at the once every 30 days or once every month intervals.

Embodiment 29: The method of any of embodiments 14-28, wherein the active material is a component of a feed and step (d) comprises instructions to administer the feed.

Embodiment 30: The method of any of embodiments 14-28, wherein the active material is a component of a chew and step (d) comprises instructions to administer the chew.

Embodiment 31: The method of any of embodiments 14-30, wherein step (c) comprises dividing the reduced dosage by about thirty (30) to yield the reduced daily dosage.

Embodiment 32: A feed, comprising an effective amount of an active material for controlling a flea and/or tick infestation in a mammal in need thereof,
wherein oral administration of the feed is continued substantially daily over a period of days to thereby increase the mammal's blood concentration of the active material to an amount effective to reduce the flea and/or tick infestation;
after the mammal's blood concentration of the active material reaches the amount effective to reduce the flea or tick infestation, the oral administration of daily feed is discontinued for a time period of at least one day, wherein the mammal's blood concentration of active material remains effective to control the flea and/or tick infestation over the time period.

Embodiment 33: The feed of embodiment 32, wherein the time period is at least three consecutive days, wherein the mammal's blood concentration of active material remains effective to control the flea and/or tick infestation over the time period.

Embodiment 34: The feed of embodiment 32, wherein the time period is at least seven consecutive days, wherein the mammal's blood concentration of active material remains effective to control the flea and/or tick infestation over the time period.

Embodiment 35: The feed of any of embodiments 32-34, further comprising resuming the administration of substantially daily feeding after the time period has elapsed and thereby continuing to maintain the mammal's blood concentration of active material in an amount effective to control the flea and/or tick infestation.

Embodiment 36: The feed of embodiment 35, wherein the time period comprises a plurality of time periods each being at least one day and all occurring within 30 days.

Embodiment 37: The feed of embodiment 32, wherein the active material is selected from the group consisting of a spinosyn and an isoxazoline.

Embodiment 38: The feed of embodiment 37, wherein the active material is a spinosyn.

Embodiment 39: The feed of embodiment 38, wherein said spinosyn is spinosad.

Embodiment 40: The feed of embodiment 37, wherein the active material is an isoxazoline.

Embodiment 41: The feed of embodiment 40, wherein the isoxazoline is selected from the group consisting of mivorilaner, fluralaner, sarolaner, afoxolaner, lotilaner, umifoxolaner, esafoxolaner, tigolaner, modoflaner, and salts thereof.

Embodiment 42: The feed of any of embodiments 32-41, wherein the feed is a wet or dry feed or a treat.

Embodiment 43: An active material for use in controlling flea and/or tick infestations in a mammal,
wherein the active material has a prescribed dosage for oral administration to a mammal for controlling fleas and/or ticks, wherein the prescribed dosage for the active material is for a prescribed administration regimen of once every 30 days or once a month and optionally repeating the prescribed administration regimen;
wherein the amount of the prescribed dosage is multiplied by about 0.90 or less to yield a reduced dosage;
wherein the reduced dosage is divided by about thirty (30) to yield a reduced daily dosage; and
wherein the active material at the reduced daily dosage is administered to the mammal substantially daily over the course of several days;
wherein, after the course of the several day administration, the mammal's flea and/or tick population is controlled to the same or greater extent than when administering the prescribed dosage of active material to the mammal at the one month or longer intervals.

Embodiment 44: The active material of embodiment 43, wherein the amount of the prescribed dosage is multiplied by between about 0.20 to about 0.90.

Embodiment 45: The active material of any of embodiments 43 and 44, wherein the amount of the prescribed dosage is multiplied by no more than about 0.70.

Embodiment 46: The active material of any of embodiments 43-45, wherein the amount of the prescribed dosage is multiplied by no more than about 0.50.

Embodiment 47: The active material of any of embodiments 43-46, wherein the active material is selected from the group consisting of a spinosyn and an isoxazoline.

Embodiment 48: The active material of embodiment 47, wherein the active material is a spinosyn.

Embodiment 49: The active material of embodiment 48, wherein said spinosyn is spinosad.

Embodiment 50: The active material of embodiment 49, wherein the amount of the prescribed dosage is multiplied by about 12.5% to 90%.

Embodiment 51: The active material of embodiment 50, wherein the prescribed dosage controls fleas and not ticks and the reduced daily dosage controls fleas and ticks.

Embodiment 52: The active material of embodiment 50, wherein the maximum concentration in the mammal's blood of the active material reaches less than about 10% of the maximum blood concentration reached when administering the prescribed dosage of active material to the mammal at the one month or longer intervals.

Embodiment 53: The active material of embodiment 47, wherein the active material is an isoxazoline.

Embodiment 54: The active material of embodiment 53, wherein the isoxazoline is selected from the group consisting of mivorilaner, fluralaner, sarolaner, afoxolaner, lotilaner, tigolaner, umifoxolaner, esafoxolaner, modoflaner, and salts thereof.

Embodiment 55: The active material of embodiment 54, wherein the active material is mivorilaner.

Embodiment 56: The active material of embodiment 55, wherein the amount of the prescribed dosage is multiplied by about 0.125 to about 0.9.

Embodiment 57: The active material of embodiment 55, wherein the maximum concentration in the mammal's blood of the active material reaches less than about 20% of the maximum blood concentration reached when administering the prescribed dosage of active material to the mammal at the one month or longer intervals.

Embodiment 58: An active material for use in controlling flea and/or tick infestations in a mammal,
wherein the active material has a prescribed dosage for oral administration to a mammal for controlling fleas and/or ticks, and wherein the prescribed dosage for the active material is for a prescribed administration regimen of once every 30 days or once every month;
wherein the amount of the prescribed dosage is multiplied by about 0.90 or less to yield a reduced dosage;
wherein the reduced dosage is converted into a reduced daily dosage; and
wherein the active material at the reduced daily dosage is to be administered to the mammal substantially daily over the course of several days;
wherein, after the course of the several day administration, the mammal's flea and/or tick population is controlled to the same or greater extent than when administering the prescribed dosage of active material to the mammal at the once every 30 days or once every month intervals.

Embodiment 59: The active material of embodiment 58, wherein the amount of the prescribed dosage is multiplied by about 0.20 to about 0.90.

Embodiment 60: The active material of any of embodiments 58-59, wherein the amount of the prescribed dosage is multiplied by no more than about 0.70.

Embodiment 61: The active material of any of embodiments 58-60, wherein the amount of the prescribed dosage is multiplied by no more than about 0.50.

Embodiment 62: The active material of any of embodiments 58-61, wherein the active material is selected from the group consisting of a spinosyn and an isoxazoline.

Embodiment 63: The active material of embodiment 62, wherein the active material is a spinosyn.

Embodiment 64: The active material of embodiment 63, wherein said spinosyn is spinosad.

Embodiment 65: The active material of embodiment 64, wherein the amount of the prescribed dosage is multiplied by about 12.5% to 90%.

Embodiment 66: The active material of embodiment 63, wherein the prescribed dosage controls fleas and not ticks and the reduced daily dosage controls fleas and ticks.

Embodiment 67: The active material of embodiment 58, wherein the active material is an isoxazoline.

Embodiment 68: The active material of embodiment 67, wherein the isoxazoline is selected from the group consisting of mivorilaner, fluralaner, sarolaner, afoxolaner, lotilaner, tigolaner, umifoxolaner, esafoxolaner, modoflaner, and salts thereof.

Embodiment 69: The active material of embodiment 68, wherein the active material is mivorilaner.

Embodiment 70: The active material of embodiment 69, wherein the amount of the prescribed dosage is multiplied by about 0.125 to about 0.9.

Embodiment 71: The active material of any of embodiments 58-70, wherein the active material is a component of a feed.

Embodiment 72: The active material of any of embodiments 58-70, wherein the active material is a component of a chew.

Embodiment 73: The active material of any of embodiments 58-72, wherein the reduced daily dosage is between 1/10 to 1/30 the amount of the reduced dosage.

Embodiment 74: The active material of embodiment 73, wherein the reduced daily dosage is between 1/15 to 1/30 the amount of the reduced dosage.

Embodiment 75: The active material of embodiment 74, wherein the reduced daily dosage is about 1/30 the amount of the reduced dosage.

In an aspect of any of the embodiments wherein the active material is an isoxazoline, administration for controlling a tick infestation provides a therapeutically effective concentration of the particular isoxazoline in said mammal's blood for at least 30 days. The precise concentration may vary according to the particular isoxazoline. For example, administration of mivorilaner provides a concentration of isoxazoline of more than about 400 ng/mL and less than about 4000 ng/mL in said mammal's blood for at least 30 days. In another example, afoxolaner provides a concentration of isoxazoline of more than about 20 ng/mL and less than about 800 ng/mL in said mammal's blood for at least 30 days. In another example, fluralaner provides a concentration of isoxazoline of more than about 40 ng/mL and less than about 2000 ng/mL in said mammal's blood for at least 30 days. In another example, sarolaner provides a concentration of isoxazoline of more than about 10 ng/mL and less than about 600 ng/mL in said mammal's blood for at least 30 days. In another example, lotilaner provides a concentration of isoxazoline of more than about 80 ng/mL and less than about 2000 ng/mL in said mammal's blood for at least 30 days. In another example, tigolaner provides a concentration of isoxazoline of more than about 10 ng/mL and less than about 600 ng/mL in said mammal's blood for at least 30 days. In another example, umifoxolaner provides a concentration of isoxazoline of more than about 10 ng/mL and less than about 600 ng/mL in said mammal's blood for at least 30 days. In another example, esafoxolaner provides a concentration of isoxazoline of more than about 10 ng/mL and less than about 400 ng/mL in said mammal's blood for at least 30 days.

In an aspect of any of the embodiments wherein the active material is an isoxazoline, administration for controlling a tick infestation provides a therapeutically effective concentration of the particular isoxazoline in said mammal's blood for at least 365 days. The precise concentration may vary according to the particular isoxazoline. For example, administration of mivorilaner provides a concentration of isoxazoline of more than about 400 ng/mL and less than about 4000 ng/mL in said mammal's blood for at least 365 days. In another example, afoxolaner provides a concentration of isoxazoline of more than about 20 ng/mL and less than about 800 ng/mL in said mammal's blood for at least 365 days. In another example, fluralaner provides a concentration of isoxazoline of more than about 40 ng/mL and less than about 2000 ng/mL in said mammal's blood for at least 365 days. In another example, sarolaner provides a concentration of isoxazoline of more than about 10 ng/mL and less than about 600 ng/mL in said mammal's blood for at least 365 days. In another example, lotilaner provides a concentration of isoxazoline of more than about 80 ng/mL and less than about 2000 ng/mL in said mammal's blood for at least 365 days. In another example, tigolaner provides a concentration of isoxazoline of more than about 10 ng/mL and less than about 600 ng/mL in said mammal's blood for at least 365 days. In another example, umifoxolaner provides a concentration of isoxazoline of more than about 10 ng/mL and less than about 600 ng/mL in said mammal's blood for at least 365 days. In another example, esafoxolaner provides a concentration of isoxazoline of more than about 10 ng/mL and less than about 400 ng/mL in said mammal's blood for at least 365 days.

In an aspect of any of the embodiments wherein the active material is an isoxazoline, administration for controlling a flea infestation provides a therapeutically effective concentration of the particular isoxazoline in said mammal's blood for at least 30 days. The precise concentration may vary according to the particular isoxazoline. For example, administration of mivorilaner provides a concentration of isoxazoline of more than about 40 ng/mL and less than about 2500 ng/mL in said mammal's blood for at least 30 days. In another example, afoxolaner provides a concentration of isoxazoline of more than about 2 ng/mL and less than about 600 ng/mL in said mammal's blood for at least 30 days. In another example, fluralaner provides a concentration of isoxazoline of more than about 4 ng/mL and less than about 1500 ng/mL in said mammal's blood for at least 30 days. In another example, sarolaner provides a concentration of isoxazoline of more than about 1 ng/mL and less than about 400 ng/mL in said mammal's blood for at least 30 days. In another example, lotilaner provides a concentration of isoxazoline of more than about 8 ng/mL and less than about 2000 ng/mL in said mammal's blood for at least 30 days. In another example, tigolaner provides a concentration of isoxazoline of more than about 1 ng/mL and less than about 300 ng/mL in said mammal's blood for at least 30 days. In another example, umifoxolaner provides a concentration of isoxazoline of more than about 1 ng/mL and less than about 300 ng/mL in said mammal's blood for at least 30 days. In another example, esafoxolaner provides a concentration of isoxazoline of more than about 1 ng/mL and less than about 300 ng/mL in said mammal's blood for at least 30 days.

In an aspect of any of the embodiments wherein the active material is an isoxazoline, administration for controlling a flea infestation provides a therapeutically effective concentration of the particular isoxazoline in said mammal's blood for at least 365 days. The precise concentration may vary according to the particular isoxazoline. For example, administration of mivorilaner provides a concentration of isoxazoline of more than about 40 ng/mL and less than about 2500 ng/mL in said mammal's blood for at least 365 days. In another example, afoxolaner provides a concentration of isoxazoline of more than about 2 ng/mL and less than about 600 ng/mL in said mammal's blood for at least 365 days. In another example, fluralaner provides a concentration of isoxazoline of more than about 4 ng/mL and less than about 1500 ng/mL in said mammal's blood for at least 365 days. In another example, sarolaner provides a concentration of isoxazoline of more than about 1 ng/mL and less than about 400 ng/mL in said mammal's blood for at least 365 days. In another example, lotilaner provides a concentration of isoxazoline of more than about 8 ng/mL and less than about 2000 ng/mL in said mammal's blood for at least 365 days. In another example, tigolaner provides a concentration of isoxazoline of more than about 1 ng/mL and less than about 300 ng/mL in said mammal's blood for at least 365 days. In another example, umifoxolaner provides a concentration of isoxazoline of more than about 1 ng/mL and less than about 300 ng/mL in said mammal's blood for at least 365 days. In another example, esafoxolaner provides a concentration of isoxazoline of more than about 1 ng/mL and less than about 300 ng/mL in said mammal's blood for at least 365 days.

In an aspect of any of the embodiments wherein the active material is a spinosyn, administration provides a concentration of spinosyn of more than about 300 ng/mL and less than about 6000 ng/mL in said mammal's blood for at least 30 days. More preferably, administration provides a concentration of spinosyn of more than about 300 ng/mL and less than about 2500 ng/mL in said mammal's blood for at least 30 days. Still more preferably, administration provides a concentration of spinosyn of more than about 300 ng/mL and less than about 2000 ng/mL in said mammal's blood for at least 30 days. Still more preferably, administration provides a concentration of spinosyn of more than about 400 ng/mL and less than about 1500 ng/mL in said mammal's blood for at least 30 days.

In an aspect of any of the embodiments wherein the active material is a spinosyn, administration provides a concentration of spinosyn of more than about 300 ng/mL and less than about 6000 ng/mL in said mammal's blood for at least 365 days. More preferably, administration provides a concentration of spinosyn of more than about 300 ng/mL and less than about 2500 ng/mL in said mammal's blood for at least 365 days. Still more preferably, administration provides a concentration of spinosyn of more than about 300 ng/mL and less than about 2000 ng/mL in said mammal's blood for at least 365 days. Still more preferably, administration provides a concentration of spinosyn of more than about 400 ng/mL and less than about 1500 ng/mL in said mammal's blood for at least 365 days.

In an aspect of any of the embodiments wherein the active material is a spinosyn, administration for controlling a flea infestation maintains a concentration of spinosyn of at least 5 ng/ml and not more than 600 ng/ml in said canine's blood for at least 30 days. More preferably, administration maintains a concentration of spinosyn of at least 5 ng/ml and not more than 300 ng/ml in said canine's blood for at least 30 days. More preferably, administration maintains a concentration of spinosyn of at least 10 ng/ml and not more than 225 ng/ml in said mammal's blood for at least 30 days. Still more preferably, administration maintains a concentration of spinosyn of at least 25 ng/ml and not more than 200 ng/ml in said mammal's blood for at least 30 days.

In an aspect of any of the embodiments wherein the active material is a spinosyn, administration for controlling a flea infestation maintains a concentration of spinosyn of at least 5 ng/ml and not more than 600 ng/ml in said canine's blood for at least 365 days. More preferably, administration maintains a concentration of spinosyn of at least 5 ng/ml and not more than 300 ng/ml in said canine's blood for at least 365 days. Still more preferably, administration maintains a concentration of spinosyn of at least 10 ng/ml and not more than 225 ng/ml in said mammal's blood for at least 365 days. Still more preferably, administration maintains a concentration of spinosyn of at least 25 ng/ml and not more than 200 ng/ml in said mammal's blood for at least 365 days.

### EXAMPLES

The following examples illustrate the methods of this disclosure:

### Example 1

Efficacy of Spinosyn Administered per os, i.e. by Mouth, to Dogs for the Treatment and Control of Rhipicephalus sanguineus.

Methods: A pool of 40 dogs are to be preliminarily infested with ˜100 unfed adult C. felis in order to produce a pool of dogs that can suitably sustain a reliable infestation rate of approximately 50% of live fleas over a 48-hour period. The dogs with the highest live flea counts are to be randomly allocated to 2 treatment groups (6 dogs per group) based on their pre-treatment flea counts from experimental infestations. The first treatment group is to be the control group and the second treatment group to be the test group.

The dogs are to be housed individually during the study period and are to be fed a commercial dry dog food ration with ad libitum access to water.

Each dog in the test group is to receive by mouth a liquid formulation of spinosyn preferably spinosad. The dosage of 2.5 mg/kg of the dog's weight is to be administered to the dogs on each of days 0-29 and the dosage of 5 mg/kg of the dog's weight is to be administered on days 30-50.

Dogs in the control group are not to receive spinosyn or any other tick control treatment. Each dog in the test group is to be offered its daily ration (dry food) and the individual doses of liquid formulation are to be administered after the individual dog has eaten at least 25% of its total daily ration. After receiving the dose of spinosyn the dogs are to be allowed to continue eating. This mimics incorporating the spinosyn in feed. Each dog in the test group and the control group is to be experimentally infested with 50 unfed adult ticks (ca. 50% male/50% female) on test days 12, 19, 28, 35, 42, 49 and 56. Comb counts for attached live and moribund adult ticks are to be conducted on days 14, 21, 30, 37, 44, 51 and 58. Note that the dosage is to be increased at day 30 and the final dose is to be given on day 50.

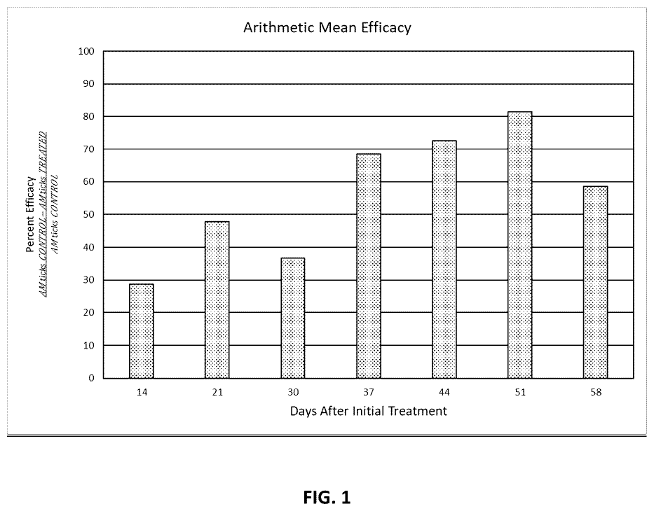

Results: Referring now to FIG. 1. Percent reduction in attached live and moribund adult tick counts for the test group treated with spinosad as compared to the control group is shown in the graph. Percent Efficacy is determined according to the formula [AM ticks CONTROL−AM ticks TREATED]/AM ticks Control. This is plotted versus time in days after initial treatment. As can be seen in FIG. 1, the daily dose is increased from 2.5 mg/kg/day to 5.0 mg/kg/day at about hour 700 (around day 30). The corresponding increase in efficacy is clear from the efficacy in days 37-50. The efficacy at day 58 is after the about 8 day wash-out period following the last dose on day 50.

Using the same study method as described above, blood is to be drawn at 72, 120, 168, 336, 504, 720 and 888 hours after the initial dose of spinosyn is administered. The average concentration of spinosyn in the blood for different dosage levels can then be determined.

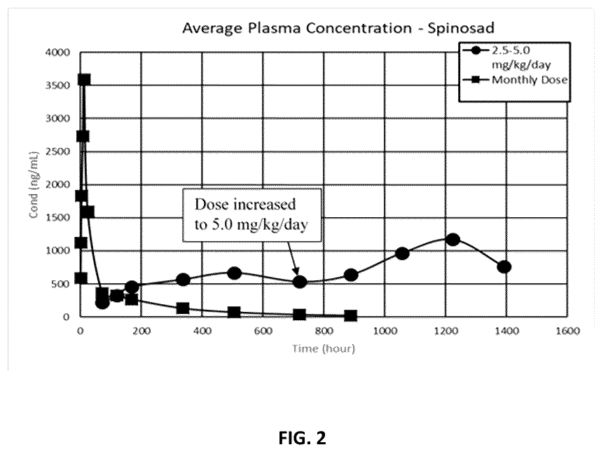

Referring now to TABLE 1 and FIG. 2. Sample results are shown for the average plasma concentration of spinosad in a canine's blood (ng/mL) versus time in hours for different dosage levels of spinosad. By way of comparison, the average plasma concentration of spinosyn for a single-dose administration to be given monthly (squares) is shown with the average plasma concentration of spinosyn for a substantially daily dose of 2.5 mg/kg/day or 5.0 mg/kg/day (circles). The daily dose is increased from 2.5 mg/kg/day to 5.0 mg/kg/day at about hour 700 (around day 30).

TABLE 1
Average Plasma Concentration (ng/ml) - Spinosad

| | 2.5-5.0 mg/kg/day | Monthly |
|---|---|---|
| Hours | spinosad | Dose |
| 1 | No Data | 585.43 |
| 2 | | 1119.50 |
| 4 | | 1830.25 |
| 8 | | 2730.50 |
| 12 | | 3592.50 |
| 24 | | 1594.50 |
| 72 | 221.50 | 359.48 |
| 120 | 327.07 | 320.25 |
| 168 | 455.78 | 265.10 |
| 336 | 566.25 | 128.95 |
| 504 | 668.77 | 69.78 |
| 720 | 533.12 | 32.23 |
| 888 | 635.72 | 20.44 |
| 1056 | 967.33 | No Data |
| 1224 | 1172.60 | |
| 1392 | 761.45 | |

### Example 2

Efficacy of Spinosyn Administered per os, i.e. by Mouth, to Dogs for the Treatment and Control of Ctenocephalides felis.

Methods: A pool of 40 dogs are to be preliminarily infested with ˜100 unfed adult C. felis in order to produce a pool of dogs that can suitably sustain a reliable infestation rate of approximately 50% of live fleas over a 48-hour period. The dogs with the highest live flea counts are to be randomly allocated to 4 treatment groups (6 dogs per group) based on their pre-treatment flea counts from experimental infestations. The first treatment group is to be the control group and groups 2-4 are to be the test groups.

The dogs are to be housed individually during the study period and are to be fed a commercial dry dog food ration with ad libitum access to water.

Each dog in test groups 2-4 is to receive by mouth a liquid formulation of spinosyn, preferably spinosad. The dosage is to be administered to the dogs on each of days 0-29 according to test groups is shown in TABLE 2.

TABLE 2

| Test Group | Oral Dose |
|---|---|
| 1 | 0 mg/kg (control group) |
| 2 | 0.25 mg/kg daily |
| 3 | 0.50 mg/kg daily |
| 4 | 1.0 mg/kg daily |

Dogs in the control group are not to receive a spinosyn or any other flea control treatment. Each dog in test groups 2-4 is to be offered its daily ration (dry food) and the individual doses of liquid formulation are to be administered after the individual dog has eaten at least 25% of its total daily ration. After receiving the dose of a spinosyn the dogs are to be allowed to continue eating. This mimics incorporating the spinosyn in feed. Each dog in test groups 2-4 and the control group is to be experimentally infested with 100 unfed adult fleas on test days −1, 5, 12, 19, 28 and 35. Comb counts for live adult fleas are to be conducted on days 2, 7, 14, 21, 30 and 37. The final experimental infestation is to occur five days after the last daily dose of spinosyn.

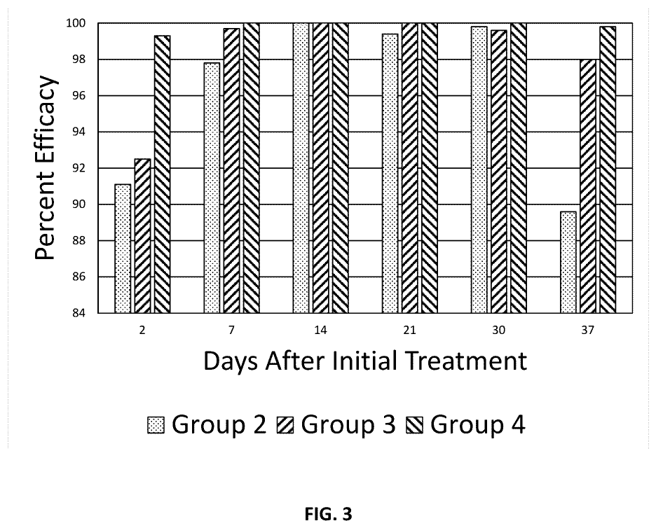

Results: Referring now to FIG. 3, it can be seen that the percent reduction in live adult flea counts for test Groups 2-4 was 100% by the day 14 comb count. Efficacy was also over 98% for Groups 3-4 on day 37, after the five day wash out period.

Using the same study method as described above, blood is to be drawn at 72, 120, 168, 336, 504, 720 and 888 hours after the initial dose of spinosyn is administered. The average concentration of spinosyn in the blood for different dosage levels can then be determined.

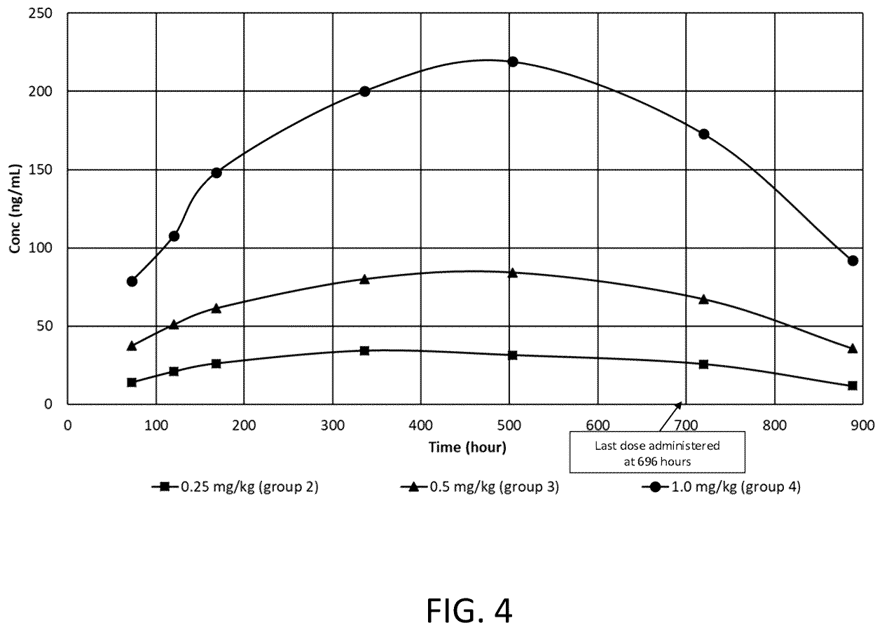

Sample results of the average plasma concentration of spinosad in a canine's blood at the different dosage levels shown in TABLE 3 and FIG. 4. Legend of FIG. 4: Group 2, 0.25 mg/kg (squares); Group 3, 0.5 mg/kg (triangles); and Group 4, 1.0 mg/kg (circles).

TABLE 3
Average Plasma concentration (ng/ml) - Spinosad

| | 0.25 | 0.5 | 1.0 | Single |
|---|---|---|---|---|
| | mg/kg/day | mg/kg/day | mg/kg/day | Monthly |
| Hours | spinosad | spinosad | spinosad | Dose |
| 1 | No Data | | | 585.43 |
| 2 | | | | 1119.50 |
| 4 | | | | 1830.25 |
| 8 | | | | 2730.50 |
| 12 | | | | 3592.50 |
| 24 | | | | 1594.50 |
| 72 | 13.99 | 37.35 | 78.71 | 359.48 |
| 120 | 20.92 | 50.97 | 107.81 | 320.25 |
| 168 | 26.07 | 61.40 | 148.10 | 265.10 |
| 336 | 34.19 | 80.06 | 200.27 | 128.95 |
| 504 | 31.38 | 84.29 | 219.00 | 69.78 |
| 720 | 25.67 | 67.21 | 172.75 | 32.23 |
| 888 | 11.73 | 35.68 | 91.82 | 20.44 |

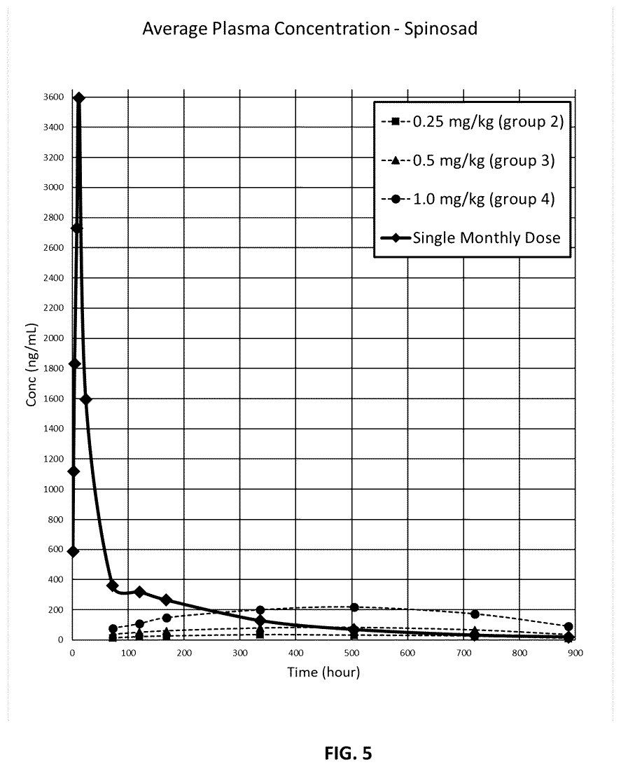

Referring now to FIG. 5, by way of comparison, the average plasma concentration of spinosad measured over time in days for a single-dose administration to be given monthly, and daily administration with one of three different levels of spinosad are shown.

### Example 3

Efficacy of Spinosyn in Dogs when Administered in a Medicated Feed Dosed at 0.5 mg/kg of the Dog's Weight.

Methods: A pool of dogs are to be preliminarily infested with ˜100 unfed adult C. felis in order to produce a pool of 18 dogs that can suitably sustain a reliable infestation rate of approximately 50% of live fleas over a 48-hour period. The dogs with the highest live flea counts are to be randomly allocated to 3 groups (6 dogs per group) based on their pre-treatment flea counts from experimental infestations. The first treatment group is to be the control group and groups 2-3 are to be the test groups.

The dogs are to be housed individually during the study period and are to have ad libitum access to water.

There is to be an initial acclimation period of at least 4 days during which dogs in test groups 2 and 3 are to be transitioned from a standard certified commercial chow to an unmedicated version of the daily feed. During the acclimation period, the dogs are to be allowed 1 hour to consume the feed, after which the dogs' acceptance of the feed will be observed and recorded.

Each dog in test groups 2 and 3 is to receive by mouth a daily feed formulation that includes spinosyn, preferably spinosad. The dosage and formulation to be administered to the dogs on each of days 0-29 according to test groups is shown in TABLE 4.

TABLE 4

| Test Group | Dose Contained in Daily Feed |
|---|---|
| 1 | 0 mg/kg (control group) |
| 2 | 0.5 mg/kg daily feed formulation 1 |
| 3 | 0.5 mg/kg daily feed formulation 2 |

Dogs in the control group are not to receive a spinosyn or any other flea control treatment. On days 0-29, each dog in test groups 2 and 3 is to be offered its daily feed containing spinosyn for a period of 1 hour. On days 30-37 all dogs will be given regular dog food, without spinosyn, or a physiologically acceptable derivative thereof

Each dog in test groups 2 and 3 and the control group is to be experimentally infested with 100 unfed adult fleas on test days -1, 5, 12, 28 and 35. Comb counts for live adult fleas are to be conducted on days 2, 7, 14, 30 and 37.

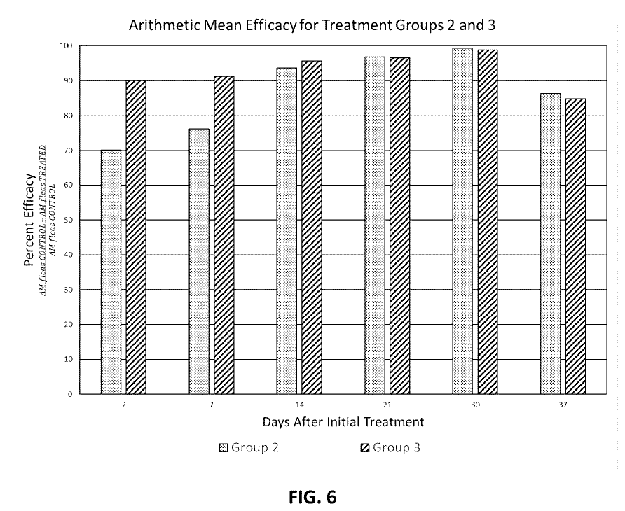

Results: In a sample study using feed containing spinosad, all dogs in Groups 2 and 3 consumed all of the food required for a 0.5 mg/kg dose without regurgitating and within the 1-hour time period. Referring now to FIG. 6, it can be seen that both treatment groups obtained over 90% efficacy by the 14-day comb count.

It can be appreciated from this example that an effective amount of a spinosyn can be administered to a dog via medicated feed to control a flea infestation even if some of the daily doses are missed.

### Example 4

Efficacy of Isoxazoline Administered per os, i.e. by mouth, to Dogs for the Treatment and Control of Rhipicephalus sanguineus.

Methods: A pool of 14 dogs are to be preliminarily infested with ˜100 unfed adult C. felis in order to produce dogs that can suitably sustain a reliable infestation rate, defined as approximately 50% fleas being live at the end of a 48-hour period. The 12 dogs with the highest live flea counts are to be selected for inclusion in the study. The dogs are to be divided into a control group and a treatment group.

The dogs are to be housed individually during the study period and are to be fed a commercial dry dog food ration with ad libitum access to water.

Each dog in the treatment group is to receive by mouth a liquid formulation of isoxazoline. The dosage of 0.75 mg/kg is to be administered to the dogs on each of days 0-29.

Dogs in the control group are not to receive isoxazoline or any other tick control treatment. Each dog in the treatment group is to be offered its daily ration (dry food) and the individual doses of liquid formulation are to be administered after the individual dog has eaten at least 25% of its total daily ration. After receiving the dose of isoxazoline, the dogs are to be allowed to continue eating. This mimics incorporating the isoxazoline in feed. Each dog in the treatment group and the control group is to be experimentally infested with 50 adult ticks on test days 5, 12, 19, 28 and 35. Comb counts for live and moribund attached ticks are to be conducted on days 7, 14, 21, 30 and 37.

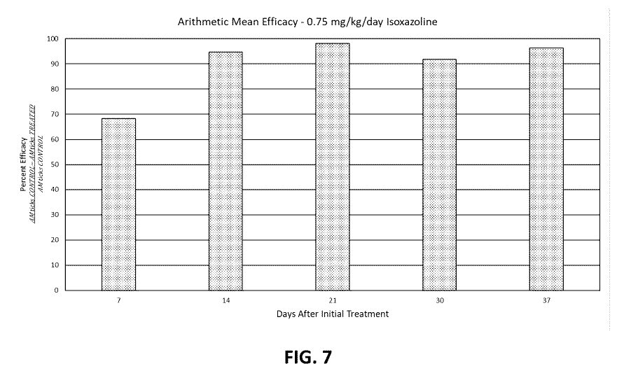

Results: Percent reduction in live and moribund attached tick counts for the treatment group treated with mivorilaner, are shown in FIG. 7.

Using the same study method as described above, blood is to be drawn at 72, 168, 336, 504, 720 and 888 hours after the initial dose of isoxazoline is administered. The average concentration of isoxazoline in the blood for different dosage levels can then be determined.

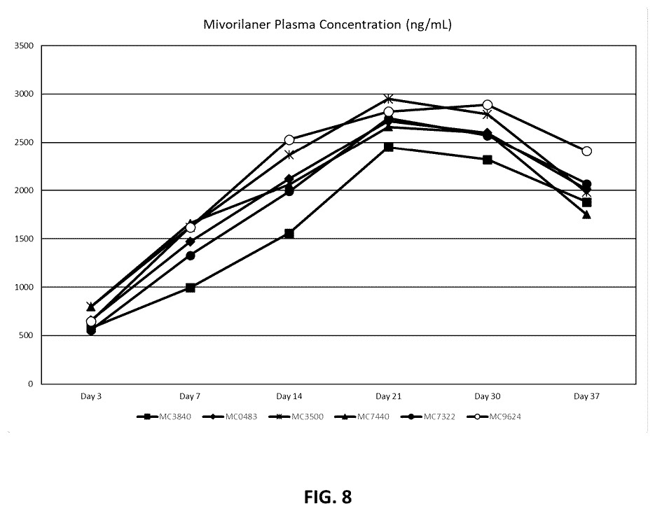

Sample results of the average plasma concentration of mivorilaner in a canine's blood at the different dosage levels are shown in TABLE 5 and FIG. 8.

TABLE 5
Mivorilaner Concentration (ng/ml)

| Canine ID | Day −1 | Day 3 | Day 7 | Day 14 | Day 21 | Day 30 | Day 37 |
|---|---|---|---|---|---|---|---|
| MC3840 | BLQ | 579 | 994 | 1560 | 2450 | 2320 | 1880 |
| MC0483 | BLQ | 660 | 1470 | 2120 | 2720 | 2600 | 2010 |
| MC3500 | BLQ | 800 | 1620 | 2370 | 2950 | 2790 | 1980 |
| MC7440 | BLQ | 798 | 1660 | 2060 | 2660 | 2600 | 1750 |
| MC7322 | BLQ | 556 | 1330 | 1990 | 2750 | 2570 | 2070 |
| MC9624 | BLQ | 648 | 1620 | 2530 | 2820 | 2890 | 2410 |
| Average | NA | 674 | 1449 | 2105 | 2725 | 2628 | 2017 |
| Std Dev | NA | 105 | 255 | 336 | 167 | 197 | 223 |
| % CV | NA | 16 | 18 | 16 | 6 | 8 | 11 |

### Example 5

Efficacy of Various Doses of Isoxazoline Administered per os, i.e. by mouth, to Dogs for the Treatment and Control of Rhipicephalus sanguineus.

Methods: A pool of 46 dogs are to be preliminarily infested with ˜50 unfed adult R. sanguineus ticks in order to produce dogs that can suitably sustain a reliable infestation rate, defined as approximately 25% of attached ticks being live at the end of a 48-hour period. The 40 dogs with the highest live attached tick counts are to be selected for inclusion in the study. The dogs are to be randomly assigned to one of a control group and 4 treatment groups.

The dogs are to be housed individually during the study period and are to be fed a commercial dry dog food ration with ad libitum access to water.

Each dog in a treatment group (test groups 2-5) is to receive by mouth a liquid formulation of isoxazoline. The dosage is to be administered to the dogs on each of days 0-59 according to test groups is listed in TABLE 6.

TABLE 6

| Treatment Group | Daily Dose (mg/kg) | Route | Fed/Fasted State |
|---|---|---|---|
| 1 (control) | 0 mg/kg | n/a | n/a |
| 2 | 0.125 mg/kg daily for | Oral | Fed |
| | 60 consecutive days | | |
| 3 | 0.25 mg/kg daily for | Oral | Fed |
| | 60 consecutive days | | |
| 4 | 0.5 mg/kg daily for | Oral | Fed |
| | 60 consecutive days | | |
| 5 | 1.0 mg/kg daily for | Oral | Fed |
| | 60 consecutive days | | |

Dogs in the control group are not to receive isoxazoline or any other tick control treatment. Each dog in the treatment group is to be offered its daily ration (dry food) and the individual doses of liquid formulation are to be administered after the individual dog has eaten at least 25% of its total daily ration. After receiving the dose of isoxazoline, the dogs are to be allowed to continue eating. This mimics incorporating the isoxazoline in feed. Each dog in treatment groups 3-5 and the control group is to be experimentally infested with 50 adult ticks on test days -2, 5, 12, 19, 28, 35 and 42. Dogs in group 2 are to be infested with 50 adult ticks on test days 19, 28 and 35. Comb counts for live adult ticks are to be conducted on days 2, 7, 14, 21, 30, 37 and 44.

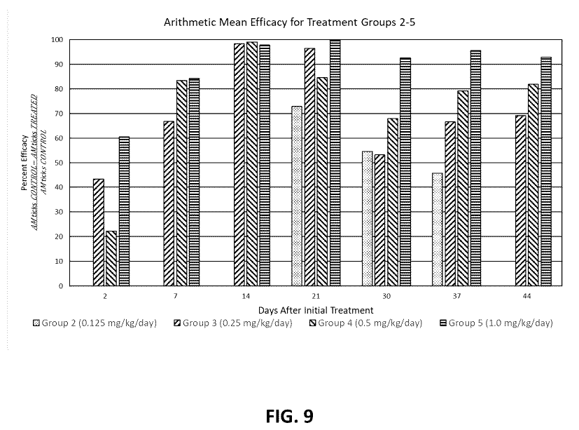

Results: Referring now to FIG. 9. Percent reduction in live and moribund attached ticks counts for the treatment groups are shown in the graph for different dosage levels of mivorilaner.

Using the same study method as described above, blood is to be drawn at 0, 72, 120, 168, 336, 504, 720, 888, 1056, 1224 and 1440 hours after the initial dose of isoxazoline is administered. The average concentration of isoxazoline in the blood for different dosage levels can then be determined.

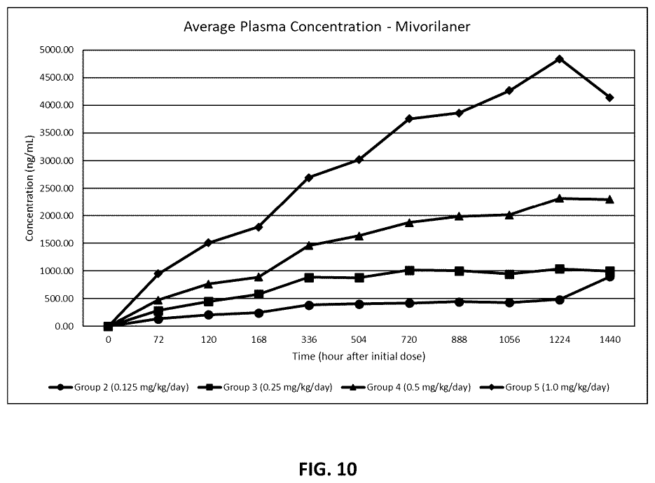

Sample results of the average plasma concentration of isoxazoline in a canine's blood at different dosage levels of mivorilaner are shown in TABLE 7 and FIG. 10.

TABLE 7
Mivorilaner Concentration (ng/mL)

| Hours | Group 2 | Group 3 | Group 4 | Group 5 |
|---|---|---|---|---|
| after | (0.125 | (0.25 | (0.5 | (1.0 |
| initial dose | mg/kg/day) | mg/kg/day) | mg/kg/day) | mg/kg/day) |
| 0 | 0.00 | 0.00 | 0.00 | 0.00 |
| 72 | 135.83 | 283.13 | 474.25 | 951.25 |
| 120 | 212.63 | 450.00 | 763.13 | 1510.00 |
| 168 | 243.63 | 579.13 | 888.00 | 1793.75 |
| 336 | 383.75 | 883.38 | 1457.88 | 2691.25 |
| 504 | 404.13 | 877.75 | 1633.25 | 3016.25 |
| 720 | 419.50 | 1015.63 | 1873.75 | 3756.25 |
| 888 | 447.50 | 1003.88 | 1986.25 | 3861.25 |
| 1056 | 428.38 | 945.13 | 2012.50 | 4263.75 |
| 1224 | 485.50 | 1040.88 | 2318.75 | 4838.75 |
| 1440 | 897.50 | 999.13 | 2298.75 | 4142.50 |

### Example 6

Comparison of Plasma Concentration of Isoxazoline in Dogs when Isoxazoline is Administered Intravenously vs. Orally in Solution and Orally in Crystal.

Methods: A pool of 24 dogs, 50% female, 50% male, 6 juveniles, 28 adults, are to be assigned to 4 study groups according to TABLE 8.

TABLE 8

| | Dose | | Dose | | Number of | |
|---|---|---|---|---|---|---|
| Group | Level | | Concentration | | Canines | |
| No. | (mg/kg) | Dose Route | (mg/mL) | Age | Male | Female |
| 1 | 0.25 | Intravenous | 0.5 | Adult | 3 | 3 |
| 2 | 0.25 | Intravenous | 0.5 | Juvenile | 3 | 3 |
| 3 | 1 | Oral Capsule | 8 mg/g | Adult | 3 | 3 |
| | | (liquid) | | | | |
| 4 | 1 | Oral Capsule | n/a | Adult | 3 | 3 |
| | | (crystal) | | | | |

Dogs are to have ad libitum access to water. On Day 1 of the study, juvenile dogs are to be offered ˜25% of their daily ration as canned feed prior to receiving the isoxazoline dose. After 4 hours, the juveniles are to be offered the remainder of their daily ration as dry feed. On day 1 of the study, adult dogs are to be provided ˜⅓ can of dog food prior to dosing and the remainder of their daily ration after the 10-hour blood collection time point. For the remainder of the study, the daily ration for all dogs should be provided for ˜2 hours.

Dogs are to receive 1 dose of isoxazoline in the fed state on Day 1 of the study. Dogs are to be fasted prior to treatment (juveniles are to be fasted <10 hours). Once it is observed that a dog has eaten 25% of its daily ration, it is to receive the isoxazoline treatment within approximately 30 minutes. This mimics incorporating the isoxazoline in feed.

Blood samples are to be taken for test groups 1 and 2 (intravenous administration) at 0, 0.083, 0.25, 0.5, 1, 3, 6, 10, 24, 48 and 96 hours after the initial treatment and 7, 10, 14, 21, 28 and 32 days after the initial treatment. Blood samples are to be taken for test groups 3 and 4 (oral administration) at 0, 0.25, 0.5, 1, 3, 6, 10, 24, 48 and 96 hours after the initial treatment and 7, 10, 14, 21, 28 and 32 days after the initial treatment. After the initial samples on day 1, dogs are to be fasted a minimum of 4 hours prior to taking further blood samples.

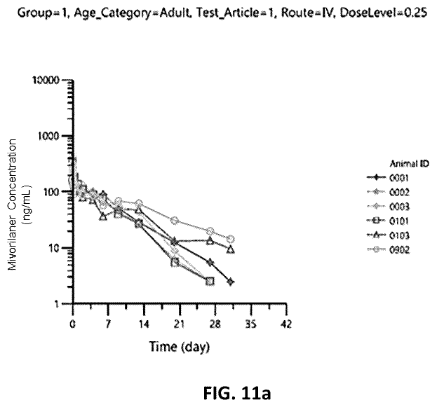

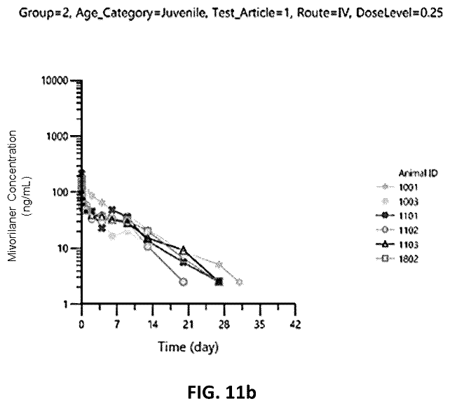

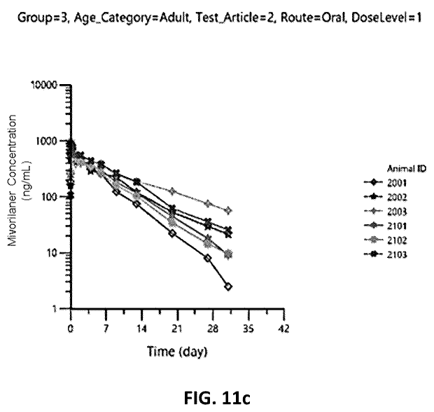

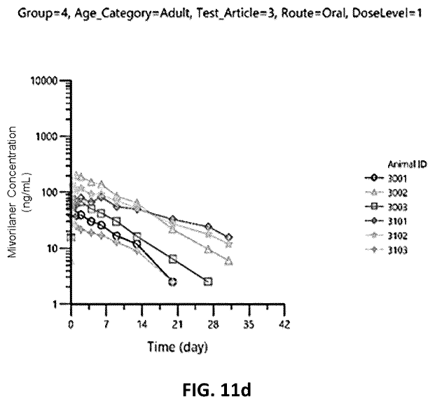

Results: The mean plasma concentrations in a study performed with mivorilaner approximately according to this example are shown in FIGS. 11a, 11b, 11c, and 11d.

### Example 7

Efficacy of Various Formulations of Isoxazoline Administered per os, i.e. by mouth, to Dogs for the Treatment and Control of Rhipicephalus sanguineus.

Methods: A pool of 36 dogs are to be preliminarily infested with ˜50 unfed adult R. sanguineus ticks in order to produce dogs that can suitably sustain a reliable infestation rate, defined as approximately 25% of attached ticks being live at the end of a 48-hour period. The 30 dogs with the highest live attached tick counts are to be selected for inclusion in the study. The dogs are to be randomly assigned to one of a control group and 4 treatment groups.

The dogs are to be housed individually during the study period and are to be fed a commercial dry dog food ration with ad libitum access to water.

Each dog in a treatment group (test groups 2-5) is to receive by mouth a liquid formulation of isoxazoline. The dosage is to be administered to the dogs on each of days according to test groups is listed in TABLE 9.

TABLE 9

| Treatment | Treatment | Anticipated Oral | |
|---|---|---|---|
| Group | Description | Dose (mg/kg) | Formulation |
| 1 | Non-treated control | 0 mg/kg | n/a |
| 2 | Afoxolaner | 0.05 mg/kg daily | Afoxolaner liquid |
| | | (Days 0-20) | formulation |
| 3 | Afoxolaner | 0.15 mg/kg daily | Afoxolaner liquid |
| | | (Days 0-20) | formulation |
| 4 | Fluralaner | 0.083 mg/kg daily | Fluralaner liquid |
| | | (Days 0-20) | formulation |
| 5 | Fluralaner | 0.25 mg/kg daily | Fluralaner liquid |
| | | (Days 0-20) | formulation |

Dogs in the control group are not to receive isoxazoline or any other tick control treatment. Each dog in the treatment group is to be offered its daily ration (dry food) and the individual doses of liquid formulation are to be administered after the individual dog has eaten at least 25% of its total daily ration. After receiving the dose of isoxazoline, the dogs are to be allowed to continue eating. This mimics incorporating the isoxazoline in feed. Each dog in the treatment group and the control group is to be experimentally infested with 50 unfed adult ticks on test days −2, 5, 12, 19 and 28. Comb counts for attached live and moribund adult ticks are to be conducted on days 2, 7, 14, 21 and 30.

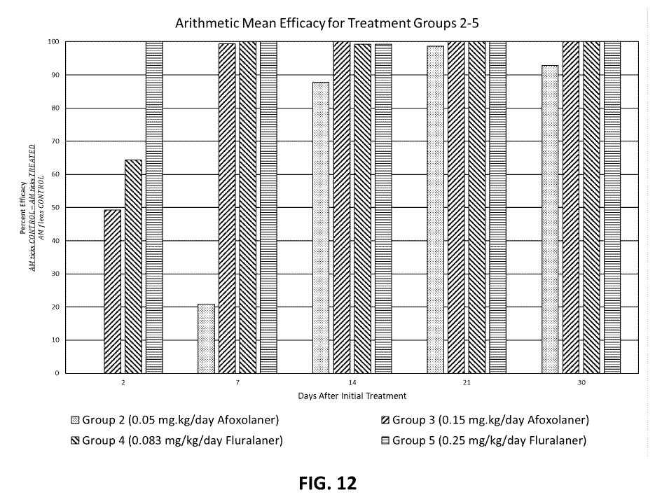

Results: Percent reduction in attached live and moribund tick counts for treatment groups approximately according to this example are shown in FIG. 12.

### Example 8

Plasma Concentration of Isoxazoline in Dogs when Isoxazoline is Administered in a Medicated Feed Dosed at 1.0 mg/kg of the Dog's Weight for One Day.

Methods: A pool of 30 dogs are to be assigned to 5 groups by weight to minimize variation between and within the groups. Each group will be given a different feed formulation containing isoxazoline and the blood level of isoxazoline over the one month period following the single dose will be determined.

The dogs are to be housed individually during the study period and are to have ad libitum access to water.

There is to be an initial acclimation period of at least 4 days during which dogs are to be transitioned from a standard certified commercial chow to an unmedicated version of the daily feed. During the acclimation period, the dogs are to be allowed 15 minutes/day to consume the feed.

On the day of the study, dogs are to be presented approximately 9.4 g/kg of daily feed containing isoxazoline. The amount of medicated feed for each dog is to be determined according to the most recent body weight of the dog prior to the day of the study. The medicated feed is to be provided for 15 minutes at the start time of the study. Any uneaten medicated feed is to be removed and weighed. An amount of unmedicated feed equaling the amount of uneaten medicated feed is to be provided ten hours later, at the first blood sampling time.

Blood samples are to be taken at the following times: 0 hr (at the time the medicated feed is provided), 0.25 hr, 0.5 hr, 1 hr, 3 hr, 6 hr, 10 hr, 1 day, 2 days, 4 days, 6 days, 9 days, 13 days, 20 days, 27 days and 31 days after the medicated feed is provided.

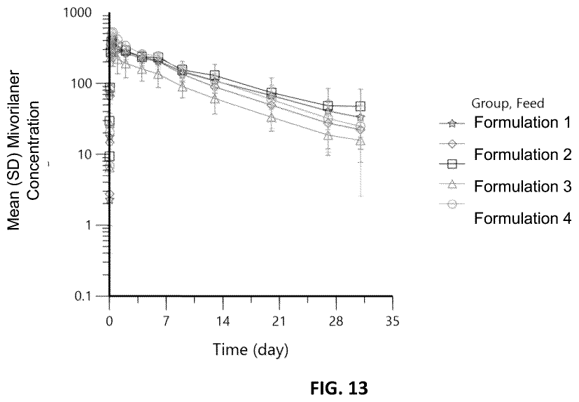

Results: The mean plasma concentrations in a study performed with mivorilaner approximately according to this example are shown in TABLE 10 and FIG. 13.

TABLE 10

| | | Target | Amount | Amount | Amount | Time to finish or | Day 1 | Mivorilaner | Dose |
|---|---|---|---|---|---|---|---|---|---|
| Canine | Group | Amount | Offered | Remaining | Consumed | bowl removed | Bodyweight | Administered | Administered |
| Number | Number: | (g) | (g) | (g) | (g) | (min) | (g) | (mg) | (mg/kg) |
| RN0001 | 1 | 66.2 | 66.0 | 0.0 | 66.0 | 5.0 | 7012.0 | 6.996 | 1.00 |
| RN0002 | | 72.1 | 72.0 | 0.0 | 72.0 | 4.0 | 7644.0 | 7.632 | 1.00 |
| RN0003 | | 71.8 | 72.0 | 25.0 | 47.0 | 15.0 | 7611.0 | 4.982 | 0.65 |
| RN0004 | | 87.7 | 88.0 | 0.0 | 88.0 | 7.0 | 9291.0 | 9.328 | 1.00 |
| RN0005 | | 89.3 | 89.0 | 0.0 | 89.0 | 6.0 | 9464.0 | 9.434 | 1.00 |
| RN0006 | | 88.9 | 89.0 | 17.0 | 72.0 | 15.0 | 9422.0 | 7.632 | 0.81 |
| RN1001 | 2 | 78.0 | 78.0 | 0.0 | 78.0 | 4.0 | 8273.0 | 8.268 | 1.00 |
| RN1002 | | 80.7 | 81.0 | 0.0 | 81.0 | 6.0 | 8552.0 | 8.586 | 1.00 |
| RN1003 | | 78.4 | 78.0 | 0.0 | 78.0 | 5.0 | 8313.0 | 8.268 | 0.99 |
| RN1004 | | 93.7 | 94.0 | 0.0 | 94.0 | 4.0 | 9934.0 | 9.964 | 1.00 |
| RN1005 | | 84.4 | 84.0 | 43.0 | 41.0 | 12.0 | 8951.0 | 4.346 | 0.49 |
| RN1006 | | 87.2 | 87.0 | 0.0 | 87.0 | 4.0 | 9247.0 | 9.222 | 1.00 |
| RN2001 | 3 | 69.6 | 70.0 | 19.0 | 51.0 | 15.0 | 7377.0 | 5.406 | 0.73 |
| RN2002 | | 65.5 | 66.0 | 66.0 | 0.0 | 16.0 | 6943.0 | 0.000 | 0.00 |
| RN2003 | | 79.6 | 80.0 | 75.0 | 5.0 | 15.0 | 8437.0 | 0.530 | 0.06 |
| RN2004 | | 90.3 | 90.0 | 3.0 | 87.0 | 15.0 | 9576.0 | 9.222 | 0.96 |
| RN2005 | | 87.1 | 87.0 | 12.0 | 75.0 | 15.0 | 9230.0 | 7.950 | 0.86 |
| RN2006 | | 98.8 | 99.0 | 71.0 | 28.0 | 15.0 | 10470.0 | 2.968 | 0.28 |
| RN3001 | 4 | 75.8 | 76.0 | 12.0 | 64.0 | 15.0 | 8033.0 | 6.784 | 0.84 |
| RN3002 | | 70.5 | 71.0 | 69.0 | 2.0 | 15.0 | 7472.0 | 0.212 | 0.03 |
| RN3003 | | 80.5 | 81.0 | 32.0 | 49.0 | 15.0 | 8538.0 | 5.194 | 0.61 |
| RN3004 | | 82.5 | 83.0 | 83.0 | 0.0 | 15.0 | 8742.0 | 0.000 | 0.00 |
| RN3005 | | 85.5 | 86.0 | 15.0 | 71.0 | 15.0 | 9067.0 | 7.526 | 0.83 |
| RN3006 | | 85.3 | 85.0 | 42.0 | 43.0 | 15.0 | 9042.0 | 4.558 | 0.50 |
| RN4001 | 5 | 70.7 | 71.0 | 71.0 | 0.0 | 17.0 | 7495.0 | 0.000 | 0.00 |
| RN4002 | | 62.3 | 62.0 | 54.0 | 8.0 | 17.0 | 6600.0 | 0.848 | 0.13 |
| RN4003 | | 66.8 | 67.0 | 0.0 | 67.0 | 6.0 | 7076.0 | 7.102 | 1.00 |
| RN4004 | | 87.8 | 88.0 | 52.0 | 36.0 | 15.0 | 9305.0 | 3.816 | 0.41 |
| RN4005 | | 94.5 | 95.0 | 0.0 | 95.0 | 6.0 | 10020.0 | 10.070 | 1.00 |
| RN4006 | | 96.8 | 97.0 | 0.0 | 97.0 | 5.0 | 10266.0 | 10.282 | 1.00 |

It can be appreciated by comparing the examples above that an effective amount of isoxazoline on average can be administered to a dog via medicated feed.

### Example 9

Efficacy of Isoxazoline when Isoxazoline is Administered in a Medicated Feed for the Treatment and Control of Rhipicephalus sanguineus.

Methods: A pool of dogs are to be preliminarily infested with ˜50 unfed adult R. sanguineus ticks in order to produce dogs that can suitably sustain a reliable infestation rate, defined as approximately 25% of attached ticks being live at the end of a 72-hour period. The 24 dogs with the highest live attached tick counts are to be selected for inclusion in the study. The 18 dogs with the highest live attached tick counts are to be randomly assigned to one of a control group and 2 treatment groups. The 6 dogs with the next highest live attached tick counts are to be assigned to a third treatment group.

The dogs are to be housed individually during the study period and are to have ad libitum access to water.

Each dog in a treatment group (test groups 2-4) is to receive a medicated daily feed from study days 0-49. The medicated daily feed is to be offered to the dogs for 1 hour on each of days 0-49 according to test groups listed in TABLE 11.

TABLE 11

| Treatment | # of | Anticipated Daily | Infestation |
|---|---|---|---|
| Group | Dogs | Dose (mg/kg) | with ticks |
| 1 | 6 | 0 | Yes |
| 2 | 6 | 1 | Yes |
| 3 | 6 | 1 | Yes |
| 4 | 6 | 3 | No |

Dogs in the control group are not to receive isoxazoline or any other tick control treatment. Each dog in treatment groups 2 and 3 and the control group is to be experimentally infested with 50 unfed adult ticks on test days −2, 4, 12 and 28 during the treatment phase and on days 52, 56 and 62 during the wash out period after the final feeding with the medicated daily feed. Comb counts for attached live and moribund adult ticks are to be conducted on days 3, 8, 15, 30, 54 and 58.

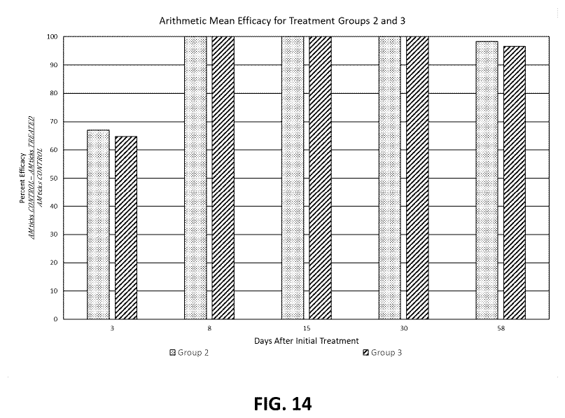

Results: Percent reduction in live adult tick counts for treatment groups approximately according to this example are shown in FIG. 14 for mivorilaner.

Using the same study method as described above, blood is to be drawn at 0, 1, 3, 6, 10, 24, 48, 96, 168, 240 and 336 hours after the initial dose of isoxazoline is administered. The average concentration of isoxazoline in the blood for different dosage levels can then be determined.

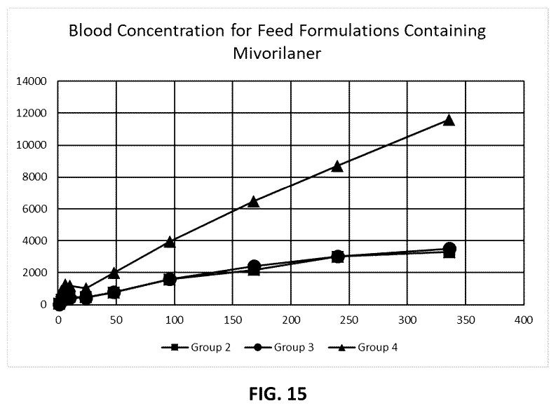

Sample results of the average plasma concentration of isoxazoline in a canine's blood at different dosage levels are shown for mivorilaner in TABLE 12 and FIG. 15.

TABLE 12

| | Group 2 - | Group 3 - | Group 4 - |
|---|---|---|---|
| Time | 106 mg/kg | 106 mg/kg | 318 mg/kg |
| (hr) | feed | feed | feed |
| 0 | | | |
| 1 | 49.98 | 40.41 | 143.75 |
| 3 | 292.00 | 264.83 | 846.50 |
| 6 | 539.00 | 467.50 | 1265.67 |
| 10 | 505.40 | 419.17 | 1198.17 |
| 24 | 458.60 | 435.17 | 1021.17 |
| 48 | 771.83 | 781.83 | 2011.67 |
| 96 | 1570.50 | 1596.67 | 3928.33 |
| 168 | 2156.67 | 2406.67 | 6461.67 |
| 240 | 3003.33 | 3026.67 | 8696.67 |
| 336 | 3290.00 | 3516.67 | 11600.00 |

### Example 10

Efficacy of Isoxazoline Administered per os, i.e. by mouth, to Dogs for the Treatment and Control of Ctenocephalides felis.

Methods: A pool of 14 dogs are to be preliminarily infested with ˜100 unfed adult C. felis in order to identify dogs that can suitably sustain a reliable infestation rate, defined as approximately 50% fleas being live at the end of a 48-hour period. The 12 dogs with the highest live flea counts are to be selected for inclusion in the study. The dogs are to be divided into a control group and a treatment group.

The dogs are to be housed individually during the study period and are to be fed a commercial dry dog food ration with ad libitum access to water.

Each dog in the treatment group is to receive by mouth a liquid formulation of isoxazoline. The dosage of 0.75 mg/kg is to be administered to the dogs on each of days 0-29.

Dogs in the control group are not to receive isoxazoline or any other flea control treatment. Each dog in the treatment group is to be offered its daily ration (dry food) and the individual doses of liquid formulation are to be administered after the individual dog has eaten at least 25% of its total daily ration. After receiving the dose of isoxazoline, the dogs are to be allowed to continue eating. This mimics incorporating the isoxazoline in feed. Each dog in the treatment group and the control group is to be experimentally infested with 100 unfed adult fleas on test days 2, 5, 12, 20 and 35. Comb counts for live adult fleas are to be conducted on days 4, 7, 14, 21 and 37.

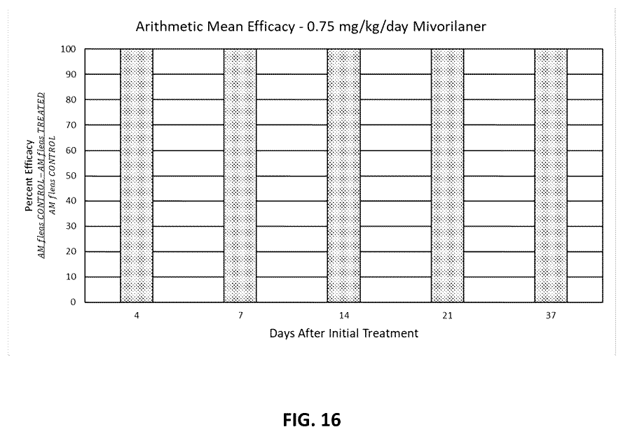

Results: referring now to FIG. 16, the arithmetic mean efficacy in reduction of live adult flea counts is shown. The Percent Efficacy is calculated according to the formula:

[AM-fleas CONTROL−AM-fleas TREATED]/AM fleas CONTROL

for a treatment group treated with 0.75 mg/kg/day of mivorilaner. As can be seen in FIG. 16, the treatment is effective on or before day 4 after initial administration.

Using the same study method as described above, blood is to be drawn at 72, 168, 336, 504, 720 and 888 hours after the initial dose of isoxazoline is administered. The average concentration of isoxazoline in the blood for different dosage levels can then be determined.

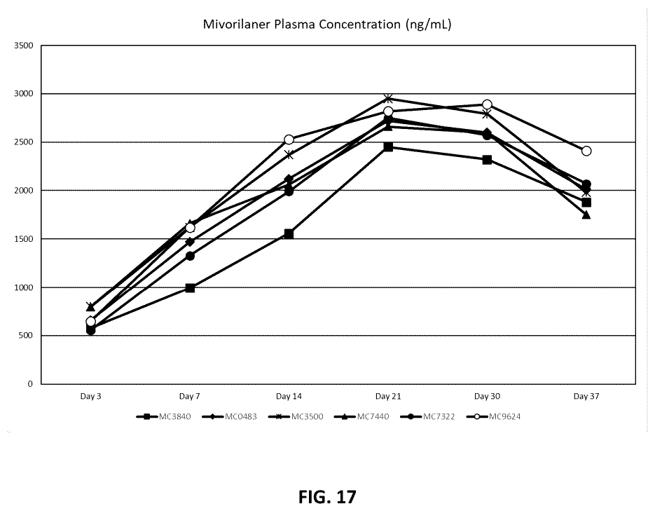

Sample results of the average plasma concentration of isoxazoline in a canine's blood at the different dosage levels shown for mivorilaner in TABLE 13 and FIG. 17.

TABLE 13
Mivorilaner Concentration (ng/mL)

| Canine ID | Day −1 | Day 3 | Day 7 | Day 14 | Day 21 | Day 30 | Day 37 |
|---|---|---|---|---|---|---|---|
| MC3840 | BLQ | 579 | 994 | 1560 | 2450 | 2320 | 1880 |
| MC0483 | BLQ | 660 | 1470 | 2120 | 2720 | 2600 | 2010 |
| MC3500 | BLQ | 800 | 1620 | 2370 | 2950 | 2790 | 1980 |
| MC7440 | BLQ | 798 | 1660 | 2060 | 2660 | 2600 | 1750 |
| MC7322 | BLQ | 556 | 1330 | 1990 | 2750 | 2570 | 2070 |
| MC9624 | BLQ | 648 | 1620 | 2530 | 2820 | 2890 | 2410 |
| Average | NA | 674 | 1449 | 2105 | 2725 | 2628 | 2017 |
| Std Dev | NA | 105 | 255 | 336 | 167 | 197 | 223 |
| % CV | NA | 16 | 18 | 16 | 6 | 8 | 11 |

### Example 11

Efficacy of Various Doses of Isoxazoline Administered per os, i.e. by mouth, to Dogs for the Treatment and Control of Ctenocephalides felis.

Methods: A pool of 46 dogs are to be preliminarily infested with ˜50 unfed adult R. sanguineus ticks in order to identify dogs that can suitably sustain a reliable infestation rate, defined as approximately 25% of attached ticks being live at the end of a 48-hour period. The 40 dogs with the highest live attached tick counts are to be selected for inclusion in the study. The dogs are to be randomly assigned to one of a control group and 4 treatment groups.

The dogs are to be housed individually during the study period and are to be fed a commercial dry dog food ration with ad libitum access to water.

Each dog in a treatment group (test groups 2-5) is to receive by mouth a liquid formulation of isoxazoline. The dosage is to be administered to the dogs on each of days 0-59 according to test groups shown in TABLE 14.

TABLE 14

| Treatment Group | Daily Dose (mg/kg) | Route | Fed/Fasted State |
|---|---|---|---|
| 1 (control) | 0 mg/kg | n/a | n/a |
| 2 | 0.125 mg/kg daily for | Oral | Fed |
| | 60 consecutive days | | |
| 3 | 0.25 mg/kg daily for | Oral | Fed |
| | 60 consecutive days | | |
| 4 | 0.5 mg/kg daily for | Oral | Fed |
| | 60 consecutive days | | |
| 5 | 1.0 mg/kg daily for | Oral | Fed |
| | 60 consecutive days | | |

Dogs in the control group are not to receive isoxazoline or any other flea control treatment. Each dog in the treatment group is to be offered its daily ration (dry food) and the individual doses of liquid formulation are to be administered after the individual dog has eaten at least 25% of its total daily ration. After receiving the dose of isoxazoline, the dogs are to be allowed to continue eating. This mimics incorporating the isoxazoline in feed. Each dog in the treatment group and the control group is to be experimentally infested with 100 unfed adult fleas on test days −1, 5, 12, 19, 28, 35 and 42. Comb counts for live adult fleas are to be conducted on days 2, 7, 14, 21, 30, 37 and 44.

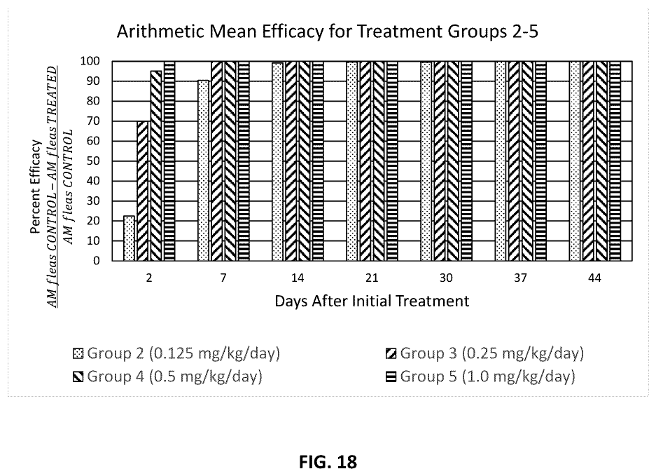

Results: Percent reduction in live adult flea counts for the treatment groups approximately according to this example are shown in FIG. 18 for mivorilaner.

Using the same study method as described above, blood is to be drawn at 0, 72, 120, 168, 336, 504, 720, 888, 1056, 1224 and 1440 hours after the initial dose of isoxazoline is administered. The average concentration of isoxazoline in the blood for different dosage levels can then be determined.

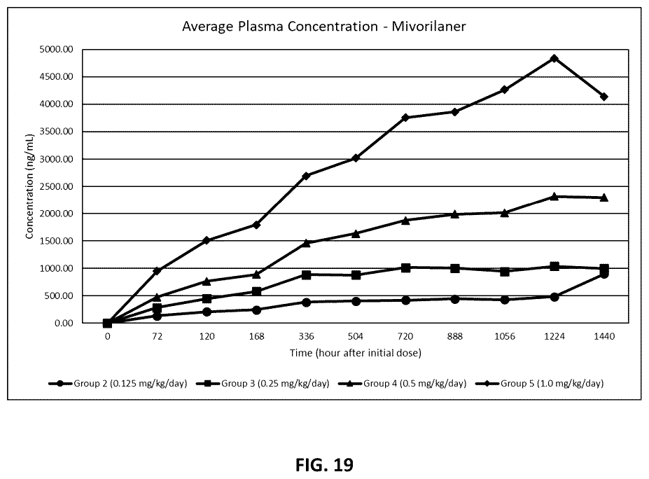

Sample results of the average plasma concentration of isoxazoline in a canine's blood at different dosage levels of mivorilaner are shown in TABLE 15 and FIG. 19.

TABLE 15
Mivorilaner Concentration (ng/mL)

| Hours | Group 2 | Group 3 | Group 4 | Group 5 |
|---|---|---|---|---|
| after | (0.125 | (0.25 | (0.5 | (1.0 |
| initial dose | mg/kg/day) | mg/kg/day) | mg/kg/day) | mg/kg/day) |
| 0 | 0.00 | 0.00 | 0.00 | 0.00 |
| 72 | 135.83 | 283.13 | 474.25 | 951.25 |
| 120 | 212.63 | 450.00 | 763.13 | 1510.00 |
| 168 | 243.63 | 579.13 | 888.00 | 1793.75 |
| 336 | 383.75 | 883.38 | 1457.88 | 2691.25 |
| 504 | 404.13 | 877.75 | 1633.25 | 3016.25 |
| 720 | 419.50 | 1015.63 | 1873.75 | 3756.25 |
| 888 | 447.50 | 1003.88 | 1986.25 | 3861.25 |
| 1056 | 428.38 | 945.13 | 2012.50 | 4263.75 |
| 1224 | 485.50 | 1040.88 | 2318.75 | 4838.75 |
| 1440 | 897.50 | 999.13 | 2298.75 | 4142.50 |

### Example 12

Efficacy of Various Formulations of Isoxazoline Administered per os, i.e. by mouth, to Dogs for the Treatment and Control of Ctenocephalides felis.

Methods: A pool of 36 dogs are to be preliminarily infested with ˜50 unfed adult R. sanguineus ticks in order to identify dogs that can suitably sustain a reliable infestation rate, defined as approximately 25% of attached ticks being live at the end of a 48-hour period. The 30 dogs with the highest live attached tick counts are to be selected for inclusion in the study. The dogs are to be randomly assigned to a control group and 4 treatment groups.

The dogs are to be housed individually during the study period and are to be fed a commercial dry dog food ration with ad libitum access to water.

Each dog in a treatment group (test groups 2-5) is to receive by mouth a liquid formulation of isoxazoline. The dosage is to be administered to the dogs on each of days 0-20 according to test groups listed in TABLE 16.

TABLE 16

| Treatment | Treatment | Anticipated Oral | |
|---|---|---|---|
| Group | Description | Dose (mg/kg) | Formulation |
| 1 | Non-treated control | 0 mg/kg | n/a |
| 2 | Afoxolaner | 0.05 mg/kg daily | Afoxolaner liquid |
| | | (Days 0-20) | formulation |
| 3 | Afoxolaner | 0.15 mg/kg daily | Afoxolaner liquid |
| | | (Days 0-20) | formulation |
| 4 | Fluralaner | 0.083 mg/kg daily | Fluralaner liquid |
| | | (Days 0-20) | formulation |
| 5 | Fluralaner | 0.25 mg/kg daily | Fluralaner liquid |
| | | (Days 0-20) | formulation |

Dogs in the control group are not to receive isoxazoline or any other flea control treatment. Each dog in the treatment group is to be offered its daily ration (dry food) and the individual doses of liquid formulation are to be administered after the individual dog has eaten at least 25% of its total daily ration. After receiving the dose of isoxazoline, the dogs are to be allowed to continue eating. This mimics incorporating the dosage in feed. Each dog in the treatment group and the control group is to be experimentally infested with 100 unfed adult fleas on test days −1, 5 and 28. Comb counts for live adult fleas are to be conducted on days 2, 7, and 30.

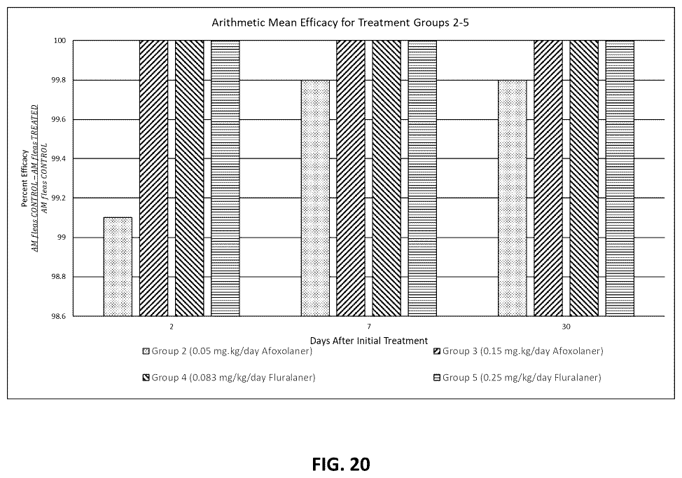

Results: Percent reduction in attached live adult flea counts for treatment groups approximately according to this example are shown in FIG. 20.

### Example 13

Efficacy of Various Formulations of Isoxazoline Administered per os, i.e. by mouth, to Dogs for the Treatment and Control of Rhipicephalus sanguineus.

Methods: A pool of 24 dogs are to be preliminarily infested with ˜50 unfed adult R. sanguineus ticks in order to identify dogs that can suitably sustain a reliable infestation rate, defined as approximately 25% of attached ticks being live at the end of a 48-hour period. The 20 dogs with the highest live attached tick counts are to be selected for inclusion in the study. The dogs are to be randomly assigned to one of a control group and 4 treatment groups.

The dogs are to be housed individually during the study period and are to be fed a commercial dry dog food ration with ad libitum access to water.

Each dog in a treatment group (test groups 2-5) is to receive by mouth a liquid formulation of isoxazoline. The dosage is to be administered to the dogs on each of days 0-27 according to test groups listed in TABLE 17.

TABLE 17

| Treatment | Treatment | Anticipated Oral | |
|---|---|---|---|
| Group | Description | Dose (mg/kg) | Formulation |
| 1 | Non-treated control | 0 mg/kg | n/a |
| 2 | Lotilaner | 0.26 mg/kg daily | Lotilaner liquid |
| | | (Days 0-27) | formulation |
| 3 | Lotilaner | 0.6 mg/kg daily | Lotilaner liquid |
| | | (Days 0-27) | formulation |
| 4 | Sarolaner | 0.016 mg/kg daily | Sarolaner liquid |
| | | (Days 0-27) | formulation |
| 5 | Sarolaner | 0.036 mg/kg daily | Sarolaner liquid |
| | | (Days 0-27) | formulation |

Dogs in the control group are not to receive isoxazoline or any other tick control treatment. Each dog in the treatment group is to be offered its daily ration (dry food) and the individual doses of liquid formulation are to be administered after the individual dog has eaten at least 25% of its total daily ration. After receiving the dose of isoxazoline, the dogs are to be allowed to continue eating. This mimics incorporating the isoxazoline in feed. Each dog in the treatment group and the control group is to be experimentally infested with 50 unfed adult ticks on test days -2, 5, 12, 19, 28 and 33. Comb counts for live and moribund attached adult ticks are to be conducted on days 2, 7, 14, 21, 30 and 35.

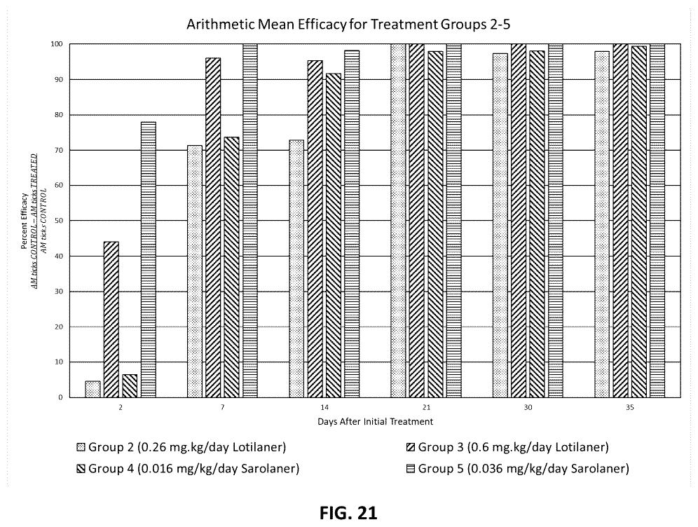

Results: Percent reduction in live and moribund attached adult tick counts for treatment groups approximately according to this example are shown in FIG. 21.

### Example 14

Efficacy of Various Formulations of Isoxazoline Administered per os, i.e. by mouth, to Dogs for the Treatment and Control of Ctenocephalides felis.

Methods: A pool of 24 dogs are to be preliminarily infested with ˜50 unfed adult Rhipicephalus sanguineus ticks in order to identify dogs that can suitably sustain a reliable infestation rate, defined as approximately 25% of attached ticks being live at the end of a 48-hour period. The 20 dogs with the highest live attached tick counts are to be selected for inclusion in the study. The dogs are to be randomly assigned to one of a control group and 4 treatment groups.

The dogs are to be housed individually during the study period and are to be fed a commercial dry dog food ration with ad libitum access to water.

Each dog in a treatment group (test groups 2-5) is to receive by mouth a liquid formulation of isoxazoline. The dosage is to be administered to the dogs on each of days 0-27 according to test groups listed in TABLE 18.

TABLE 18

| Treatment | Treatment | Anticipated Oral | |
|---|---|---|---|
| Group | Description | Dose (mg/kg) | Formulation |
| 1 | Non-treated control | 0 mg/kg | n/a |
| 2 | Lotilaner | 0.26 mg/kg daily | Lotilaner liquid |
| | | (Days 0-27) | formulation |
| 3 | Lotilaner | 0.6 mg/kg daily | Lotilaner liquid |
| | | (Days 0-27) | formulation |
| 4 | Sarolaner | 0.016 mg/kg daily | Sarolaner liquid |
| | | (Days 0-27) | formulation |
| 5 | Sarolaner | 0.036 mg/kg daily | Sarolaner liquid |
| | | (Days 0-27) | formulation |

Dogs in the control group are not to receive isoxazoline or any other flea control treatment. Each dog in the treatment group is to be offered its daily ration (dry food) and the individual doses of liquid formulation are to be administered after the individual dog has eaten at least 25% of its total daily ration. After receiving the dose of isoxazoline, the dogs are to be allowed to continue eating. This mimics incorporating the isoxazoline in feed. Each dog in the treatment group and the control group is to be experimentally infested with 100 unfed adult fleas on test days −1, 5 and 33. Comb counts for live adult fleas are to be conducted on days 2, 7, and 35.

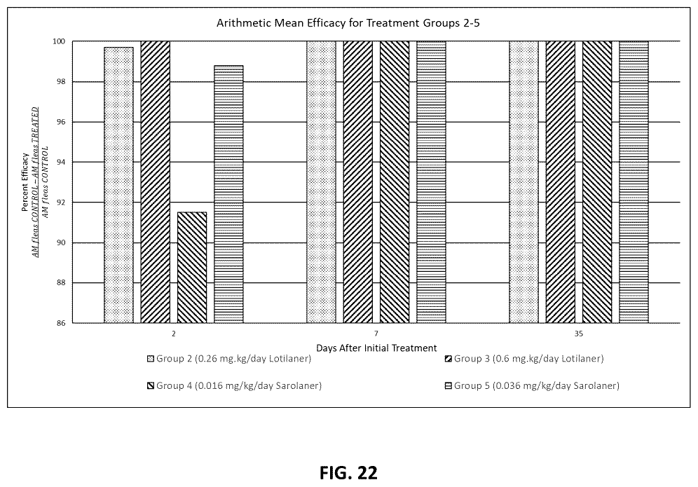

Results: Referring now to FIG. 22, the arithmetic mean efficacy in reduction of live adult flea counts is shown. The Percent Efficacy is calculated according to the formula:

[AM-fleas CONTROL−AM-fleas TREATED]/AM fleas CONTROL

for Group 2, 0.26 mg/kg/day Lotilaner (stars); Group 3, 0.6 mg/kg/day Lotilaner (right hash); Group 4, 0.016mg/kg/day Sarolaner (right hash); and Group 5, 0.036 mg/kg/day Sarolaner (horizontal hash). As can be seen in FIG. 22, all tested isoxazolines demonstrated efficacy.

While this invention has been described as having an exemplary design, the present invention may be further modified within the spirit and scope of this disclosure. This application is therefore intended to cover any variations, uses, or adaptations of the invention using its general principles.

What is claimed is:

1. A method of establishing a regimen for orally administering a reduced dosage of an active material for controlling flea and/or tick infestations in a mammal, the method comprising:
(a) selecting an active material having a prescribed dosage for oral administration to a mammal for controlling fleas and/or ticks, wherein the prescribed dosage is for a prescribed administration regimen of once every 30 days or once every month;
(b) multiplying the amount of the prescribed dosage by about 0.90 or less to yield a reduced dosage;
(c) converting the reduced dosage into a reduced daily dosage; and
(d) providing instructions to administer the active material at the reduced daily dosage to the mammal substantially daily over the course of several days;
whereby, after the instructions to administer are followed, the mammal's flea and/or tick infestation is controlled to the same or greater extent than when administering the prescribed dosage of active material to the mammal at the once every 30 days or once every month intervals.

2. The method of claim 1, wherein step (b) comprises multiplying the amount of the prescribed dosage by between about 0.20 to about 0.90.

3. The method of claim 1, wherein step (b) comprises multiplying the amount of the prescribed dosage by no more than about 0.70.

4. The method of claim 1, wherein step (b) comprises multiplying the amount of the prescribed dosage by no more than about 0.50.

5. The method of claim 1, wherein the active material is a spinosyn.

6. The method of claim 5, wherein said spinosyn is spinosad.

7. The method of claim 5, wherein step (b) comprises multiplying the amount of the prescribed dosage by about 12.5% to 90%.

8. The method of claim 7, wherein the prescribed dosage controls fleas and not ticks and the reduced daily dosage controls fleas and ticks.

9. The method of claim 5, wherein performing steps (a)-(d) results in the maximum concentration in the mammal's blood of the active material reaching less than about 10% of the maximum blood concentration reached when administering the prescribed dosage of active material to the mammal at the once every 30 days or once every month intervals.

10. The method of claim 1, wherein the active material is an isoxazoline.

11. The method of claim 10, wherein the isoxazoline is selected from the group consisting of mivorilaner, fluralaner, sarolaner, afoxolaner, lotilaner, tigolaner, umifoxolaner, esafoxolaner, modoflaner, and salts thereof.

12. The method of claim 11, wherein the active material is mivorilaner.

13. The method of claim 12, wherein step (b) comprises multiplying the amount of the prescribed dosage by about 0.125 to about 0.9.

14. The method of claim 13, wherein performing steps (a)-(d) results in the maximum concentration in the mammal's blood of the active material reaching less than about 20% of the maximum blood concentration reached when administering the prescribed dosage of active material to the mammal at the once every 30 days or once every month intervals.

15. A method of controlling a flea and/or tick infestation in a mammal in need thereof, comprising:
orally administering to said mammal an effective amount of an active material;
continuing the oral administration substantially daily over a period of days to thereby increase the mammal's blood concentration of the active material to an amount effective to reduce the flea and/or tick infestation;
after the mammal's blood concentration of the active material reaches the amount effective to reduce the flea or tick infestation, discontinuing the oral administration for a time period of at least one day, wherein the mammal's blood concentration of active material remains effective to control the flea and/or tick infestation over the time period.

16. The method according to claim 15, wherein the active material is a spinosyn.

17. The method according to claim 15, wherein the active material is an isoxazoline.

18. A method of administering an effective amount of an active material for controlling a flea and/or tick infestation in a mammal in need thereof, comprising:
orally administering a feed comprising the active material substantially daily over a period of days to thereby increase the mammal's blood concentration of the active material to an amount effective to reduce the flea and/or tick infestation; and
after the mammal's blood concentration of the active material reaches the amount effective to reduce the flea or tick infestation, discontinuing the oral administration of daily feed for a time period of at least one day, wherein the mammal's blood concentration of active material remains effective to control the flea and/or tick infestation over the time period.

19. The method according to claim 18, wherein the active material is a spinosyn.

20. The method according to claim 18, wherein the active material is an isoxazoline.
